SEQUENCE LISTING (11 sequences)

SEQ ID NO: 1 - protein, 4 aa, synthetic construct
WHHW

SEQ ID NO: 2 - protein, 4 aa, synthetic construct
WHWH

SEQ ID NO: 3 - protein, 4 aa, synthetic construct
HWWH

SEQ ID NO: 4 - protein, 4 aa, synthetic construct
HWHW

SEQ ID NO: 5 - protein, 7 aa, synthetic construct
XWHHWXX

SEQ ID NO: 6 - protein, 6 aa, synthetic construct
XWHHWX

SEQ ID NO: 7 - protein, 4 aa, synthetic construct
WAAW

SEQ ID NO: 8 - protein, 4 aa, synthetic construct
AHHA

SEQ ID NO: 9 - protein, 4 aa, synthetic construct
FHHF

SEQ ID NO: 10 - protein, 4 aa, synthetic construct
YHHY

SEQ ID NO: 11 - protein, 6 aa, synthetic construct
XWHHWX

(19) United States
(12) Patent Application Publication
(10) Pub. No.: US 2026/0297127 A1
(43) Pub. Date: Oct. 1, 2026

(54) REVERSIBLE SURFACTANT AND MULTIFUNCTIONAL DRUG DELIVERY SYSTEMS BASED THEREON

(71) Applicant: Yissum Research Development Company of the Hebrew University of Jerusalem Ltd., Jerusalem (IL)

(72) Inventors: Meital RECHES, Kfar Bin Nun (IL); Daniel BOAS, Rishon LeZion (IL)

(73) Assignee: Yissum Research Development Company of the Hebrew University of Jerusalem Ltd., Jerusalem (IL)

(21) Appl. No.: 19/482,846

(22) PCT Filed: May 23, 2024
(86) PCT No.: PCT/IL2024/050507

§ 371 Date: Nov. 10, 2025

Related U.S. Application Data
(60) Provisional application No. 63/468,693, filed on May 24, 2023.

(51) Int. Cl. C07K 5/117

(52) U.S. Cl. CPC C07K 5/1024

(57) ABSTRACT

The invention includes reversible surfactant and emulsions containing same.

### TECHNOLOGICAL FIELD

The invention generally contemplates reversible surfactants and delivery platforms based thereon.

### BACKGROUND

Emulsions are colloids of two immiscible liquids in which one liquid contains a dispersion of the other. Several types of emulsions are known and used in a variety of industrial fields. The main types of emulsions include: an oil in water (O/W) emulsion in which an oil phase is dispersed in an aqueous phase; a water in oil (W/O) emulsion in which an aqueous phase is dispersed in an oil phase; and other more complex emulsions, such as oil in water in oil, or water in oil in water.

Emulsions do not typically demonstrate a static internal structure and are thermodynamically unstable as both the dispersed and continuous phases can revert to separate phases by fusion or the coalescing of droplets. To stabilize emulsions, emulsifying agents such as surfactants are utilized. Hydrophilic surfactants support O/W emulsions, whereas lipophilic surfactants support W/O emulsions.

### GENERAL DESCRIPTION

Emulsion drug delivery systems are a common method for administering lipophilic drugs. They can be easily prepared and applied, and they have been extensively investigated. However, these systems are limited to the delivery of either lipophilic drugs dissolved in a non-aqueous phase of an oil-in-water (o/w) emulsion, or water-soluble drugs that are provided in water-in-oil (w/o) emulsions. Emulsions that contain both lipophilic and hydrophilic drugs are not possible. The separation between the drugs is detrimental to the potential of emulsions in drug delivery, as it negates possible synergetic therapeutic effects that water-soluble and water-insoluble compounds may have. Such a synergistic therapy could originate from combinations of lipophilic and hydrophilic drugs, combinations of drugs and stimuli-responsive materials or combinations of drugs with metal ions.

Several anti-tumor drugs can be taken as an example for this problem. The efficacy of the chemotherapeutic drug Paclitaxel (PTX) was shown to be enhanced by the presence of Zn²⁺ ions. Similarly, the anti-tumor effects of disulfiram and curcumin were also shown to be promoted by interactions with metal ions. Some of these compounds which activity is enhanced by presence of metal ions are lipophilic and cannot be dissolved in water, prohibiting their delivery alongside metal ions.

To achieve simultaneous delivery of both water-soluble and water-insoluble compounds, the inventors of the technology disclosed herein designed a unique methodology by which short molecular structures, such as, but not limited to peptides with metal-binding moieties are caused to change their conformation upon complexation with certain metal ions and adopt amphiphilicity not characteristic of the uncomplexed form. The induced or triggered amphiphilicity allows emulsification of both lipophilic and hydrophilic agents in an aqueous or oil medium, generating stable emulsions such as water-in-oil (W/O) or oil-in-water (O/W) emulsions.

An exemplary system is depicted in FIG. 1A.

The structural or conformational change can be viewed as a molecular switch that allows to control the material properties, as it turns the non-amphiphilic material into an amphiphilic complex. Unlike Gemini surfactants, in which two amphiphilic materials (same or different) are connected at their polar ends to fix the amphiphilic nature of the material, materials of the invention are not amphiphilic in their uncomplexed conformation, and their pH-triggered or metal-ion triggered amphiphilicity is reversible.

Thus, in a first aspect, there is provided a non-amphiphilic compound comprising one or more metal binding moieties and one or more hydrophobic functionalities, said compound being reversibly convertible into an amphiphilic compound upon exposure to metal ions (e.g., via binding of the one or more metal binding moieties to a metal ion or a metal ion containing material).

Typically, a compound having a single metal binding moiety and a single hydrophobic functionality is amphiphilic, and therefore excluded from the scope of the present invention.

The invention further provides a metal ion-activated molecular switch having a non-amphiphilic conformation and an amphiphilic conformation, wherein said amphiphilic conformation is reversibly triggered in presence of a metal ion or a metal ion containing material.

Further provided is a compound switchable between amphiphilic and non-amphiphilic conformations, the compound comprising one or more metal-binding moieties and one or more hydrophobic functionality, wherein binding of a metal ion to the one or more metal binding moieties triggers a reversible conformational change from the non-amphiphilic conformation to the amphiphilic conformation.

Further provided is a compound for emulsifying both a lipophilic agent and a metal-based hydrophilic agent, said compound being switchable between amphiphilic and non-amphiphilic conformations, wherein in the amphiphilic conformation the compound establishes a chemical association between the metal-based hydrophilic agent and one or more metal binding moieties of the compound, the compound further establishes an interaction between the lipophilic agent and one or more hydrophobic functionality of the compound; the stability between the amphiphilic and non-amphiphilic conformations being metal-ion based.

The invention further provides a compound having triggerable and reversible amphiphilicity, the compound comprising one or more metal binding moieties and one or more hydrophobic functionalities and configured, in an aqueous medium comprising metal ions, to emulsify a lipophilic agent and a hydrophilic agent.

In some embodiments of compounds of the invention, the compounds comprise two or more metal binding moieties.

Compounds of the invention are typically single-compound molecular switches having a stable non-amphiphilic conformation, whereby presence of a metal ion induces conformational change and formation of distinct hydrophobic and hydrophilic regions. As used herein, a “conformational change” generally refers to change in a spatial arrangement or rearrangement of atoms or groups of atoms or functionalities in the molecule that result from rotation of its constituent groups of about bonds. Compounds of the invention may be generally characterized as having at least one stable spatial arrangement that is non-amphiphilic, and which is, in some cases, the thermodynamically preferred conformation. Conformational conversion from a non-amphiphilic conformation to an amphiphilic conformation and formation of the hydrophobic and hydrophilic regions results from the reorientation of the compound metal-binding moieties in space, responsive to the presence of the metal ion, and as such the conformational change may be triggered by any means increasing the metal ion content of a medium containing the compound. Such means may be addition or presence of metal salts or compounds having a metallic ion, increasing pH, etc. The reorientation of the metal-binding moieties results in the repositioning of the hydrophobic groups opposite to the binding groups. Unlike common surfactants, which have distinct hydrophobic and hydrophilic regions, compounds of the invention are not characterized as amphiphilic, unless in presence of a metal ion. A non-limiting depiction of compounds or molecular switches according to the invention includes non-amphiphilic conformations, as follows:

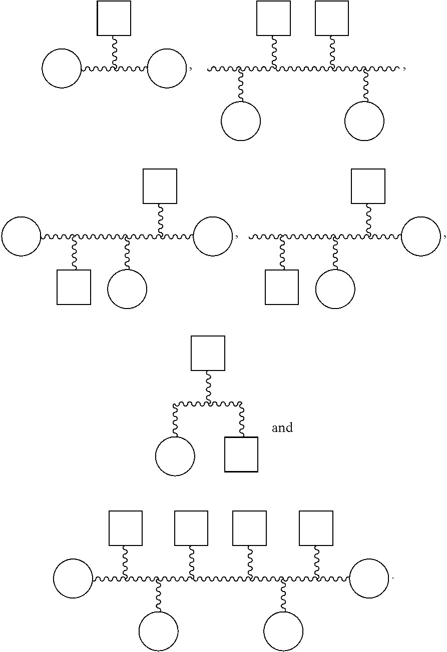

Each of the circles designates a metal-binding moiety and each of the squares designates a hydrophobic functionality. As exemplified, each of the compounds are non-amphiphilic as each adopts a conformation which lacks distinct hydrophobic/hydrophilic regions. In presence of a metal ion, however, complexation with the metal-binding moieties induces the conformation change and induction of amphiphilicity, as depicted below:

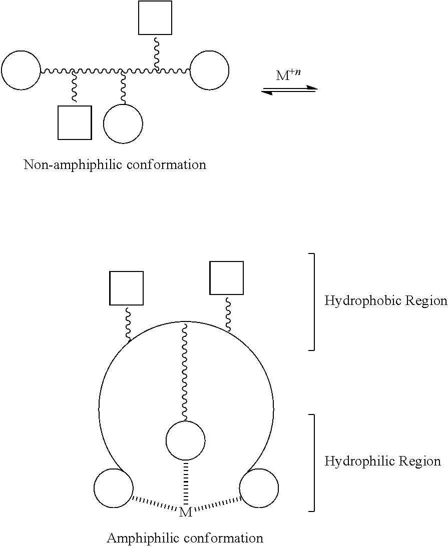

As known in the art, a material is considered “amphiphilic” or having “amphiphilicity” if the material has distinct hydrophobic (or lipophilic) and hydrophilic regions. For a material to be amphiphilic, it has to demonstrate a dual affinity: towards water, through its hydrophilic region and to oil through its hydrophobic region. Compounds of the invention, in their non-amphiphilic or uncomplexed conformations do not exhibit such a dual affinity.

When referring to “water”, it should be understood that the term encompasses water and any aqueous formulation or any water-based formulation.

Compounds of the invention are typically structured around a short backbone having between 5 and 10 backbone atoms (typically carbon atoms with, optionally, one or more interrupting heteroatoms). The compound may be a linear or branched aliphatic compound, comprising a backbone having between 5 and 10 carbon atoms and substituted by one, two or more metal binding moieties and one or more hydrophobic groups. Additional side groups and substituents may also be present. Alternatively, the backbone may comprise amino acids or be a short peptide, formed of between 2 and 10 amino acids, and comprising one, two or more metal-binding moieties and one or more hydrophobic functionalities. Some or all of the metal-binding moieties and/or hydrophobic functionalities may be inherent to certain amino acids making-up the peptide.

In order to maximize freedom to undergo conformational changes, the compounds are typically not heavily substituted with steric groups. Where such groups are present, they may be positioned such that conformational changes are not hindered or prevented.

One or two or more metal-binding moieties are present on each compound. The moieties may be same or different, provided that they are similarly capable of associating with a metal ion of interest. In some cases, the moieties are the same. The position of the moieties along the backbone of the compound is not restricted. However, for certain applications, the moieties may be substituted at the ends (termini) of the compound backbone and any additional moieties may be substituted along the backbone, as generally depicted above.

The metal-binding moieties may be selected amongst any chemical group capable of associating to a metal ion. The association is not limited. The type of moieties selected may depend on the metal to be associated, the metal charge, ligand groups that may be present on the metal, and other considerations known in the art. Generally, the metal may be any metal ion or any metal-containing material (e.g., complex). The metal ion or complex may be of a metal used in medicine, diagnosis, cosmetics, agriculture, printing, lithography, in the food industry, in the beverage industry, etc. The metals may be selected amongst monovalent metals, bivalent metal, trivalent metals and metals of higher oxidation states. Non-limiting examples of metals include copper (Cu⁺¹, Cu⁺²), calcium (Ca⁺²), chromium (Cr⁺², Cr⁺³), magnesium (Mg⁺²), iron (Fe⁺², Fe⁺³), zinc (Zn⁺²), platinum (Pt⁺², Pt⁺⁴), molybdenum (Mo⁺², Mo⁺³, Mo⁺⁴), technetium (Tc⁺⁴), vanadium (V⁺², V⁺³), manganese (Mn⁺², Mn⁺³), nickel (Ni⁺²), thallium (Tl⁺³), indium (In⁺²), silver (Ag⁺¹), gold (Au⁺³), cobalt (Co⁺²), aluminum (Al⁺², Al⁺³), ytterbium (Yb³⁺), europium (Eu³⁺), praseodymium (Pr³⁺), Gadolinium (Gd³⁺), and others.

In some embodiments, the metal atom is an active isotope, e.g., of a transition metal, such as those used in medicine or diagnosis, e.g., imaging.

In some embodiments, the metal ions are provided in salt forms, generating complexes with the metal-binding moieties. In some embodiments, the metal ions are provided in a complex form with one or more active organic or inorganic or hybrid moiety, generating a complex with the metal-binding moieties, wherein the active moiety acts as a ligand. The active moiety may be any group of material functionality containing a metal ion, including, for example, nano or microparticles having ligands with metal-binding capabilities that are coordinated with metal ions, protein drugs, antibodies, chemical drugs presented in salt forms, contrast agents presented with a metal ion, and others.

As used herein, the “metal-binding moiety” is at least one organic group that comprises an atom or a group of atoms (which may be different from carbon) capable of reversibly associating (and dissociating) to a metal ion baring any valency or charge. The functionality is selected amongst such which are capable of holding the metal ion and releasing it under predetermined conditions. The functionality may be selected to hold the metal ion under certain conditions and release the metal ion once such conditions are altered. The conditions may be a change of pH, ionic strength, presence or absence of better chelating agents, thermal conditions, and others. The term may refer to any one of the groups or to a combination of two or more of the groups, e.g., amino acids, which are capable of binding, coordinating or associating with the metal ion atom or group.

The compound of the invention may be a peptide structured of two or more amino acids, at least one or two of which are metal-binding amino acids and one or more of the amino acids is a hydrophobic amino acid. In other words, compounds of the invention may be dipeptides, tripeptides, tetrapeptides, pentapeptides, hexapeptides or longer peptides having 7 or more amino acids. In some embodiments, the peptide comprises 2, 3, 4, 5, 6, 7, 8, 9, or 10 amino acids. In some embodiments, in a peptide of the invention, the amino acids are associated to each other via peptide bonds. In other cases, the amino acids may be associated through a linker atom or a linker group.

In some embodiments, the compound is a peptide having 4, 5, 6 or 7 amino acids. In some embodiments, the compound is a tetrapeptide. Each peptide constitutes a separate invention.

Compounds of the invention, being in some embodiments, peptides, may comprise any amino acid. The term “amino acid” as used throughout the present application, unless otherwise specified, refers to any natural or unnatural amino acid, an amino acid analog, α- or β-forms, or may be in either L- or D configurations. Amino acid analogs which may be used in a compound of the invention may be chemically modified at either or both C-terminal and/or N-terminal; or chemically modified at a side-chain functional group (e.g., positioned at the α-position or any other pendant group).

The amino acid may be selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selenocysteine; and amino acid analogs such as homo-amino acids, N-alkyl amino acids, dehydroamino acids, aromatic amino acids and α,α-disubstituted amino acids, e.g., cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In some embodiments, the amino acids are selected amongst aromatic amino acids. Non-limiting examples of aromatic amino acids include histidine, tryptophan, tyrosine, naphthylalanine, and phenylalanine.

In some embodiments, a compound of the invention is a peptide, which may or may not be symmetric in structure. The peptide may be a symmetric peptide, whereby each of the metal binding amino acids are positioned at the termini of the peptide and wherein the hydrophobic amino acids are provided between the metal-binding amino acids at central region of the peptide chain. Alternatively, the two metal-binding amino acids are provided between the two hydrophobic amino acids, which are provided at the termini of the peptide. Exemplary symmetric peptides are provided above and reproduced below:

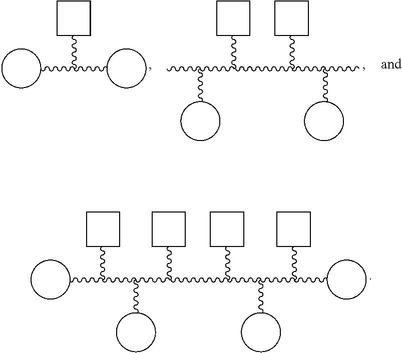

The peptides may alternatively be asymmetric. Exemplified structures are provided above and reproduced below:

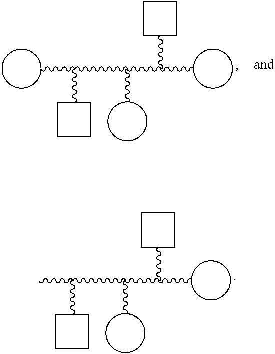

Additional symmetric and asymmetric peptides are also possible.

Suitable metal-binding moieties may include an atom selected from sulfur, nitrogen, oxygen, phosphorus, and others, each being associated or bonded to an organic linker or group. In some embodiments, the atom is provided as a group of same or different atoms.

In some embodiments, the metal-binding moiety is at least one organic group comprising one or more sulfur atoms, and/or nitrogen atoms, and/or oxygen atoms, and/or phosphorus atoms. In some embodiments, the organic group is an amino acid or a peptide.

In some embodiments, the metal-binding amino acid is selected amongst such amino acids having at least one pendent nitrogen group. Non-limiting examples include histidine, arginine, proline and lysine.

In some embodiments, the amino acid is histidine.

In some embodiments, the peptide comprises 2 or more histidine units. In some embodiments, a histidine unit is provided at each terminus, and wherein optionally at least one another histidine is provided along the peptide chain.

In some embodiments, the number of histidine units is between 2 and 5, 2 and 4, 3 and 5, or 2, 3, 4 or 5. In some embodiments, the number of histidine units is 2.

In some embodiments, the metal-binding moiety is derived from ethylenediaminetetraacetic acid (EDTA), nitrilotriacetic acid (NTA), pyridine and amino terminal Cu(II)- and Ni(II)-binding (ATCUN) motifs.

In another aspect, there is provided a non-amphiphilic peptide comprising two or more histidine binding moieties and one or more hydrophobic functionalities, said peptide being reversibly convertible into an amphiphilic peptide upon exposure to metal ions (e.g., via binding of the two or more histidine units to a metal ion or a metal ion containing material).

The invention further provides a peptide having a non-amphiphilic conformation and an amphiphilic conformation, wherein said amphiphilic conformation is reversibly triggered in presence of a metal ion or a metal ion containing material.

Further provided is a peptide switchable between amphiphilic and non-amphiphilic conformations, the peptide comprising two or more histidine units and one or more hydrophobic functionality, wherein binding of a metal ion to the two or more histidine units triggers a reversible conformational change from the non-amphiphilic conformation to the amphiphilic conformation.

The one or more “hydrophobic functionality” may be any such chemical group which is substantially water-insoluble but is soluble in an oil phase; namely wherein the solubility in the oil phase is higher than that in water or in an aqueous phase. In some embodiments, the hydrophobic group may be a hydrocarbon having at least 6 carbon atoms, an aromatic group or a large chemical group having low or no solubility in water.

In some embodiments, the hydrophobic functionality is a hydrophobic amino acid, such as glycine, alanine, valine, leucine, isoleucine, proline, phenylalanine, methionine, and tryptophan. In some embodiments, the hydrophobic amino acid is tryptophan.

In some embodiments, the peptide comprises 1 or more tryptophan units. In some embodiments, a tryptophan unit is provided at a peptide terminus, and wherein optionally at least one another tryptophan is provided along the peptide chain.

In some embodiments, the number of tryptophan units is between 1 and 4, 1 and 3, 2 and 4, or 1, 2, 3, or 4. In some embodiments, the number of tryptophan units is 2.

In some embodiments, compounds of the invention are peptides having one or more histidine units (acting at metal-binding moieties) and one or more tryptophan units (acting as hydrophobic functionalities).

In some embodiments, a peptide of the invention comprises two or more histidine units (acting at metal-binding moieties) and two or more tryptophan units (acting as hydrophobic functionalities).

In some embodiments, a peptide of the invention comprises two histidine units (acting at metal-binding moieties) and two tryptophan units (acting as hydrophobic functionalities).

In some embodiments, a peptide of the invention is a tetrapeptide comprising two histidine units (acting at metal-binding moieties) and two tryptophan units (acting as hydrophobic functionalities).

In some embodiments, the peptide is of the general formula Y—X₁X₂X₃X₄—Y₁, wherein each of X₁, X₂, X₃ and X₄, independently, is an amino acid, wherein two of said amino acids are metal-binding amino acids, as selected herein, and two of the remaining amino acids are hydrophobic amino acids, as selected herein; and wherein each of Y and Y₁ is absent or selected amongst amino acids, as disclosed herein. In some embodiments, one or both of Y and Y₁ are absent.

In some embodiments, the metal-binding amino acid is histidine and the hydrophobic amino acid is tryptophan. Non-limiting examples of peptides of the invention include Trp-His-His-Trp (SEQ ID NO 1), Trp-His-Trp-His (SEQ ID NO 2), His-Trp-Trp-His (SEQ ID NO 3), His-Trp-His-Trp (SEQ ID NO 4).

In some embodiments, the metal binding amino acid is histidine and the hydrophobic amino acid is phenylalanine or naphthylalanine or a derivative thereof.

In another aspect, there is provided a non-amphiphilic peptide comprising one or more histidine amino acids and one or more tryptophan amino acid, said peptide being reversibly convertible into an amphiphilic peptide upon exposure to metal ions (e.g., via binding of the one or more histidine units to a metal ion or a metal ion containing material).

Further provided is a peptide switchable between amphiphilic and non-amphiphilic conformations, the peptide comprising one or more histidine amino acids and one or more tryptophan amino acids, wherein binding of a metal ion to the two or more histidine amino acids triggers a reversible conformational change from the non-amphiphilic conformation to the amphiphilic conformation.

Further provided is a peptide switchable between amphiphilic and non-amphiphilic conformations, the peptide comprising two histidine amino acids and two tryptophan amino acids, wherein binding of a metal ion to the two histidine amino acids triggers a reversible conformational change from the non-amphiphilic conformation to the amphiphilic conformation.

Amphiphilic compounds of the invention comprise a metal complex region (hydrophilic in nature) and a hydrophobic region (defined by the one or more hydrophobic functionalities of the compound). These compounds reduce the surface tension between water and oil and the interfacial tension between a liquid and a solid. As such, compounds of the invention may be used “reversible surfactants” or “conditional surfactants” that are capable, in presence of a metal ion, to modify surface and interface properties of liquids or solids; e.g., emulsification, stability, spreading, wetting, dispersibility, or other surface properties. In absence of metal ions, the compounds are not amphiphilic.

Thus, the invention further provides a reversible surfactant being a compound of the invention.

The surfactants may be presented as self-assembled fibrous structure, or as spherical structures when in presence of metal ions. In presence of a hydrophobic or oil phase, e.g., the lipophilic agent, the surfactants may be formed into micelles. The surfactants of the invention may thus be provided in a form of a powder, in a solution form (as an aqueous solution), as a fibrous material (in water), as a spheres, as an emulsion or as a micellar material.

The invention further provides a surface modifying material consisting or comprising a compound of the invention, wherein the material is capable of modifying a surface property of a liquid or a solid in the presence of metal ion(s).

In some embodiments, the surface property is emulsification, stability, spreading, wetting, or dispersibility in a water or an oil medium. Thus, the material of the invention may be used as a surfactant, a detergent, a wetting agent, an emulsifier, a solubilizing agent, a dispersing agent, and a foaming agent, in a variety of fields including medicine, cosmetics, agriculture and generally in the material sciences.

The amphiphilic nature of compounds of the invention has been exploited for emulsifying two materials in a water-oil interface: a lipophilic material that undergoes a stabilizing interaction with the oil phase and a hydrophilic material that is stabilized by the water or aqueous phase. The nature of the emulsion is not limited. Compounds of the invention may be used in water-in-oil (W/O) emulsions, oil-in water (O/W) emulsions, as well as in W/O/W and O/W/O emulsions.

The two agents emulsified in the presence of the surfactant of the invention are a metal-base hydrophilic agent that is solubilized in the aqueous or water phase of the emulsion and a lipophilic agent that constitutes the oil phase of the emulsion or is comprised by the oil phase. Where the lipophilic agent is comprised by the oil phase, the oil carrier constituting the oil phase may be any suitable fatty or oily material that is insoluble in water and which may be inert. The oil phase thus may contain the lipophilic agent and at least one oil carrier. The aqueous or water phase may similarly comprise in addition to the metal-based hydrophilic agent one or more additional water-soluble materials, additive, etc. Thus, a water-oil mixture, e.g., in a form of an emulsion, may comprise water or an aqueous component which comprises the metal-based hydrophilic agent and an oil component, which may be the lipophilic agent, and a surfactant according to the invention, emulsifying both agents.

The invention further provides a water-oil mixture (wherein water encompasses any aqueous composition) comprising a lipophilic agent, a metal-based hydrophilic agent and a reversible surfactant comprising at least one metal-binding moiety and at least one hydrophobic functionality,
the reversible surfactant being switchable between an amphiphilic conformation and a non-amphiphilic conformation, whereby association between the at least one metal-binding moiety of the reversible surfactant and a metal ion of the metal-based hydrophilic agent induces emulsification of both the lipophilic agent and the metal-based hydrophilic agent,
wherein dissociation of the metal-based hydrophilic agent from the reversible surfactant triggers loss of amphiphilicity and release of the hydrophilic and lipophilic agents.

The dissociation of the metal-based hydrophilic agent may be achievable under any of the conditions disclosed herein, e.g., a change in pH, a change in the ionic strength, a change in the temperature, etc. The dissociation causes loss of amphiphilicity as the conformation of the reversible surfactant revers to non-amphiphilic. This reversion causes separation of the lipophilic agent and the metal-based hydrophilic agent and increases their simultaneous availability to the water-oil mixture or its environment, e.g., tissue in case of emulsified drugs.

The water-oil mixture is typically an emulsion wherein the at least one metal-binding moiety of the surfactant associates said metal-based hydrophilic agent at an interface between an aqueous phase comprising the metal-based hydrophilic agent and an oil phase comprising or being the lipophilic agent.

The invention therefore provides an emulsion comprising a compound or surfactant according to the invention provided at an interface between an aqueous phase and an oily phase (being a W/O or O/W or W/O/W or O/W/O emulsion), the compound being associated to a metal ion or a metal-containing material (the hydrophilic material).

Also provided is a peptide-stabilized emulsion, as disclosed herein, wherein the compound or surfactant is a peptide as defined herein.

Further provides is an emulsion comprising a lipophilic agent, a metal-based hydrophilic agent and an amphiphilic surfactant comprising at least one metal-binding moiety and at least one hydrophobic functionality,
the at least one metal-binding moiety of the amphiphilic surfactant associating said metal-based hydrophilic agent at an interface between an aqueous phase comprising the metal-based hydrophilic agent and an oil phase comprising or being the lipophilic agent,
wherein dissociation of the metal-based hydrophilic agent from the amphiphilic surfactant triggers loss of amphiphilicity and release of the hydrophilic and lipophilic agents, as disclosed and explained herein.

In some embodiments, the oily phase consists or comprises an active, e.g., a lipophilic active. Thus, the emulsion comprising the compound or surfactant of the invention comprises both a lipophilic active and a hydrophilic active. The two actives may be selected amongst pharmaceutically active agents, cosmetically active agents, agriculturally active agents, food additives, dietary additives, and others.

In some embodiments, the two actives are pharmaceutically acceptable drugs, medicaments or agents used in medicine; namely capable of inducing treatment or prevention of a disease or disorder. Such drugs may be selected amongst cytotoxic drugs or chemotherapeutic drugs, anticancer drugs, anti-inflammatory drugs, antimicrobial drugs, antioxidants, antihistamines, hormones, analgesics, vitamins, anthelmintic, anti-arrhythmic drugs, anti-coagulants, anti-depressants, anti-diabetics, anti-diarrheal, anti-epileptics, anti-hypertensive agents, anti-malarial, anti-migraine agents, anti-muscarinic agents, immunosuppressants, anti-protozoal agents, anti-rheumatics, anti-thyroid agents, anxiolytics, sedatives, hypnotics and neuroleptics, antipsychotics, beta-blockers, cardiac inotropic agents, corticosteroids, cough suppressants, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents, oral vaccines, proteins, peptides and recombinant drugs, sex hormones and contraceptives, spermicides, stimulants, and combinations thereof.

The lipophilic and hydrophilic pharmaceutical active agent may be selected amongst representative drugs including, for example:
Anti-inflammatory drugs: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and others;
Antimicrobial drugs (including antibacterial, antifungal, antiprotozoal and antiviral agents): ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, miconazole, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate, clotrimazole and others;
Antioxidants: ascorbic acid (vitamin C), ascorbyl esters of fatty acids, ascorbic acid derivatives, tocopherol (vitamin E), tocopherol sorbate, tocopherol acetate, 6-hydroxy-2,5,7,8-tetramethylchroman-2-carboxylic acid, silymarin, and others;
Antihistamines: chlorpheniramine, brompheniramine, dexchlorpheniramine, tripolidine, clemastine, diphenhydramine, promethazine, piperazines, piperidines, astemizole, loratadine, terfenadine and others;
Hormones: methyltestosterone, androsterone, androsterone acetate, androsterone propionate, androsterone benzoate, androsteronediol, androsteronediol-3-acetate, androsteronediol-17-acetate, androsteronediol 3,17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone and others;
Analgesics: acetaminophen, alfentanil hydrochloride, aminobenzoate potassium, aminobenzoate sodium, anidoxime, anileridine, anileridine hydrochloride, anilopam hydrochloride, anirolac, antipyrine, aspirin, benoxaprofen, benzydamine hydrochloride, bicifadine hydrochloride, brifentanil hydrochloride, bromadoline maleate, bromfenac sodium, buprenorphine hydrochloride, butacetin, butixirate, butorphanol, butorphanol tartrate, carbamazepine, carbaspirin calcium, carbiphene hydrochloride, carfentanil citrate, ciprefadol succinate, ciramadol, ciramadol hydrochloride, clonixeril, clonixin, codeine, codeine phosphate, codeine sulfate, conorphone hydrochloride, cyclazocine, dexoxadrol hydrochloride, dexpemedolac, dezocine, diflunisal, dihydrocodeine bitartrate, dimefadane, dipyrone, doxpicomine hydrochloride, drinidene, enadoline hydrochloride, epirizole, ergotamine tartrate, ethoxazene hydrochloride, etofenamate, eugenol, fenoprofen, fenoprofen calcium, fentanyl citrate, floctafenine, flufenisal, flunixin, flunixin meglumine, flupirtine maleate, fluproquazone, fluradoline hydrochloride, flurbiprofen, hydromorphone hydrochloride, ibufenac, indoprofen, ketazocine, ketorfanol, ketorolac tromethamine, letimide hydrochloride, levomethadyl acetate, levomethadyl acetate hydrochloride, levonantradol hydrochloride, levorphanol tartrate, lofemizole hydrochloride, lofentanil oxalate, lorcinadol, lornoxicam, magnesium salicylate, mefenamic acid, menabitan hydrochloride, meperidine hydrochloride, meptazinol hydrochloride, methadone hydrochloride, methadyl acetate, methopholine, methotrimeprazine, metkephamid acetate, mimbane hydrochloride, mirfentanil hydrochloride, molinazone, morphine sulfate, moxazocine, nabitan hydrochloride, nalbuphine hydrochloride, nalmexone hydrochloride, namoxyrate, nantradol hydrochloride, naproxen, naproxen sodium, naproxol, nefopam hydrochloride, nexeridine hydrochloride, noracymethadol hydrochloride, ocfentanil hydrochloride, octazamide, olvanil, oxetorone fumarate, oxycodone, oxycodone hydrochloride, oxycodone terephthalate, oxymorphone hydrochloride, pemedolac, pentamorphone, pentazocine, pentazocine hydrochloride, pentazocine lactate, phenazopyridine hydrochloride, phenyramidol hydrochloride, picenadol hydrochloride, pinadoline, pirfenidone, piroxicam olamine, pravadoline maleate, prodilidine hydrochloride, profadol hydrochloride, propiram fumarate, propoxyphene hydrochloride, propoxyphene napsylate, proxazole, proxazole citrate, proxorphan tartrate, pyrroliphene hydrochloride, remifentanil hydrochloride, salcolex, salethamide maleate, salicylamide, salicylate meglumine, salsalate, sodium salicylate, spiradoline mesylate, sufentanil, sufentanil citrate, talmetacin, talniflumate, talosalate, tazadolene succinate, tebufelone, tetrydamine, tifurac sodium, tilidine hydrochloride, tiopinac, tonazocine mesylate, tramadol hydrochloride, trefentanil hydrochloride, trolamine, veradoline hydrochloride, verilopam hydrochloride, volazocine, xorphanol mesylate, xylazine hydrochloride, zenazocine mesylate, zomepirac sodium, zucapsaicin and others;
Anticancer drugs: Chlorambucil; 3-(9-Acridinylamino)-5-(hydroxymethyl) aniline; Azatoxin; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Disulfiram; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, and others;
Chemotherapeutic drugs: camptothecin, daunorubicin, doxorubicin, N-(5,5-diacetoxypentyl) doxorubicin, anthracycline, mitomycin C, mitomycin A, 9-amino aminopertin, antinomycin, N8-acetyl spermidine, 1-(2-chloroethyl)-1,2-dimethanesulfonyl hydrazine, bleomycin, tallysomucin, etoposide, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine, vinblastine, vinca alkaloid, an epipodophyllotoxin, a taxane, anthracycline, bleomycin, doxorubicin, paclitaxel, 4-OH cyclophosphamide, cis-platin and others.

In some embodiments, the two actives may be selected amongst cosmetically active agents. The cosmetically acceptable agents may be used for inducing treatment or prevention of a skin disease or disorder, for improving skin condition or for affecting human skin, including hair and nails.

The cosmetically active agents may be selected amongst skin lightening agents, darkening agents, anti-acne agents, shine control agents, antimicrobial agents (including antifungal and antibacterial agents), anti-inflammatory agents, sunscreens, photo-protectors, antioxidants, keratolytic agents, nutrients, vitamins, energy enhancers, astringents, deodorants, hair growth enhancing agents, anticallous agents, fluorides, odor-control agents and others.

The lipophilic and the metal-based hydrophilic cosmetically acceptable agents may be selected from avobenzone, bisdisulizole disodium, diethylamino hydroxybenzoyl hexyl benzoate, ecamsule, methyl anthranilate, 4-aminobenzoic acid, cinoxate, ethylhexyl triazone, 4-methylbenzylidene camphor, phenylbenzimidazole sulfonic acid, polysilicone-15, trolamine salicylate, octocrylene, oxybenzone, titanium dioxide, zinc oxide, carotenoids, retinol, retinoic acid, ceramides, estrogens, steroids, copper salts, sugar amines, vitamin K, castor oil, squalene, lanolin, and others.

In some embodiments, the lipophilic and metal-based hydrophilic actives may be selected amongst agriculturally active agents suitable for application onto a plant part or a vicinity of a plant. The plant may be any plant known in the art, including cultivated plants, such as cereals, e.g., wheat, rye, barley, triticale, oats or rice; beet, e.g., sugar beet or fodder beet; fruits, such as apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee or tea plants; vines; forestry plants; flowering plants; trees and evergreens; bushes, etc.

In some embodiments, the actives may be selected from antimicrobial materials (including antibacterial, antiviral, antifungal, etc), insecticidal materials, insect-repelling materials, nutrients, fertilizers, pesticides, acaracides, herbicides, nemacides, rodenticides, entomopathogens, pheromones, attractants, plant growth regulators, plant hormones, insect growth regulators, chemosterilants, microbial pest control agents, phagostimulents, biological control agents, any combination of the aforementioned and others.

Non-limiting examples of lipophilic and the metal-based hydrophilic agriculturally acceptable agents include fertilizers such as urea, ammonium sulfate, ammonium nitrate, phosphate, phosphate salts, potassium chloride, magnesium nitrate, manganese nitrate, zinc nitrate, copper nitrate, phosphoric acid, potassium nitrate, potassium sulfate, calcium sulfate, calcium chloride and boric acid; pesticides such as azoxystrobin, bitertanol, carboxin, cymoxanil, cyproconazole, cyprodinil, dichlofluamid, difenoconazole, diniconazole, epoxiconazole, fenpiclonil, fludioxonil, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, metconazole, oxadixyl, pefurazoate, penconazole, pencycuron, prochloraz, propiconazole, pyroquilone, SSF-109, spiroxamin, tebuconazole, thiabendazole, tolifluamid, triazoxide, triadimefon, triadimenol, triflumizole, triticonazole and uniconazole; plant hormone such as 1-naphthalene acetic acid, 2,4,5-trichlorobenzoic acid, 2,4,5-trichlorophenoxyacetic acid, 2,4-dichlorobenzoic acid, 2,4-dichlorophenoxyacetic acid, 2-hydroxybenzoic acid, 4-chlorobenzoic acid, 4-chlorophenoxyacetic acid, 4-nitrobenzoic acid, abscisic acid, citric acid, gibberellic acid, gibberellin A13, gibberellin A3, gibberellin A4, gluconic acid, indole-3-acetic acid, indole-3-butanoic acid, oxalic acid, or salicylic acid; abscisic acid (PGH); indole-3-acetic acid, 4-chloroindole-3-acetic acid, 2-phenylacetic acid, indole 3-butanoic acid, indole-3-propanoic acid, gibberellins such as GA1, gibberellic acid (GA3), GA4, GA5, GA6, GA7, GA13, and gibberellic acid.

The lipophilic and the metal-based hydrophilic agents may be selected amongst acceptable food additive or dietary ingredients that are suitable for human or animal consumption. The food additive may be any such agent listed on the United States Food and Drug Administration (FDA) Food Additive Status List, herein incorporated by reference. The food additive may be natural, artificial or processed. The additives may be preservatives, coloring agents, flavoring agents, sweeteners, stabilizers, antioxidants, antimicrobial agents, nutrients, minerals, supplements, leavening agents, acids, acidity regulators, anticaking agents, antifoaming agents, bulking agents, color retention agents, glazing agents, humectants, thickeners and others.

Non-limiting examples of food additives include benzoate salts, calcium propionate, cured sodium erythorbate, sodium nitrite, calcium sorbate, potassium sorbate, BHA, BHT, EDTA, tocopherols, vitamins, sorbitol, mannitol, corn syrup, and others.

The dietary supplements or ingredients may be any approved agent used for supplementing a human or animal diet, or specifically for stimulating, improving or increasing general energy performance, concentration and physical stress resistance, improving the mood in situations of stress to reduce physical and mental fatigue, improve activity, improve recovery from activity, promote muscle performance, increase energy substrates etc.

The dietary supplements may be any of the food additives disclosed herein, including also vitamins, flavonoids and bioflavonoids, carotenoids, phytosterols, polyphenols, carbohydrates, proteins; including for example NADH, L-camitine, co-enzyme Q10, L-carnosine, succinic acid, ascorbic acid, and others.

A person versed in the art would know how to identify and distinguish between lipophilic agents and hydrophilic agents (pharmaceutically, cosmetic, agriculturally acceptable, food additives or dietary supplements). The terms “hydrophilic” and “lipophilic” used in reference to water solubility of agents mentioned herein have the meaning acceptable in the art. For example, metal-based hydrophilic agents may be defined as having a water solubility that is 1 g of the agent in less than 10 L, or less than 1 L, or less than 100 mL, or less than 30 mL, or less than 10 mL of water at a given pH (at around 25° C.). The metal-based hydrophilic agents may alternatively be characterized as having a negative log P. The lipophilic agents may have a water solubility that is 1 g of the agent in more than 10 L, or a log P that is positive.

In some embodiments, the metal-based hydrophilic agent is a metal salt acceptable in the various fields of use. The metal salt may be a salt used in medicine, cosmetics, imaging, agriculture, printing, material sciences, food industry and others. The metal salt may be a salt of copper (Cu⁺¹, Cu⁺²), calcium (Ca⁺²), chromium (Cr⁺², Cr⁺³), magnesium (Mg⁺²), iron (Fe⁺², Fe⁺³), zinc (Zn⁺²), platinum (Pt⁺², Pt⁺⁴), molybdenum (Mo⁺², Mo⁺³, Mo⁺⁴), technetium (Tc⁺⁴), vanadium (V⁺², V⁺³), manganese (Mn⁺², Mn⁺³), nickel (Ni⁺²), thallium (Tl⁺³), indium (In⁺²), silver (Ag⁺¹), gold (Au⁺³), cobalt (Co⁺²), aluminum (Al⁺², Al⁺³), ytterbium (Yb³⁺), europium (Eu³⁺), praseodymium (Pr³⁺), Gadolinium (Gd³⁺), and others. The salt may be derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

The metal-based hydrophilic agent may also be an organic material comprising one or more metal salt as a cation or a coordination metal. Such materials may be selected as above.

Emulsions comprising a compound of the invention with the lipophilic and the hydrophilic agents may be triggered to release the two agents; namely, to render both agents available in the water-oil mixture resulting from the collapse of the emulsion, by causing dissociation of the metal-based hydrophilic material from the metal-binding moieties. This may be achievable by changing one or more parameters or characteristics of the emulsion, such may include pH, ionic strength, presence or absence of better chelating agents, thermal conditions, and others.

This suggests that a synergistic release of the lipophilic and hydrophilic agents can be used for achieving a synergistic therapy or synergistic effect when administering the two agents at the same time, rendering both available for uptake or for inducing a concerted effect on a substrate, e.g., a tissue, a product, etc.

The invention further provides a system for delivering both a lipophilic agent and a hydrophilic agent (simultaneously, or at the same time, e.g., for achieving a synergistic release thereof), the system comprises a compound or surfactant according to the invention provided at an interface between an aqueous phase and an oily phase being or comprising a lipophilic agent, the compound being associated to a hydrophilic metal-containing material.

The system may be a formulation for delivering the actives. The formulation may be a pharmaceutical formulation for delivery of pharmaceutically active agents, drugs, etc; a cosmetic formulation for delivering onto a skin region drugs and other active agents; an agricultural formulation for delivering nutrients, plant management agents, etc; a food additive formulation for delivering a food additive to at least one food product or beverage.

In some embodiments, the system may be used for delivering nano or microparticles. The nano or microparticles may have surface groups enabling association with the metal binding moieties. The surface groups may be metal ions or metal binding moieties that are bound to metal ions, which can also interact with the metal binding moieties present on compounds or surfactants of the invention. The nano or microparticles may be formed of any material, e.g., may be polymeric, organic, inorganic or hybrid particles. The nano or microparticles may comprise one or more active or non-active material. Additionally or alternatively, the particles may be selected amongst functional particles such as conductive nanoparticles, insulator nanoparticles, metallic nanoparticles, magnetic nanoparticles, catalyst nanoparticles, optically-active nanoparticles, hard or soft nanoparticles and others.

In some embodiments, the nano or microparticles encapsulate or contain one or more active material selected as herein amongst pharmaceutically, cosmetically, agriculturally acceptable agents, food additives, and others.

In some embodiments, the nano or microparticles are selected amongst metallic particles, conductive particles, insulator particles, magnetic particles (ferromagnetic, paramagnetic, etc), catalyst particles, optically-active particles, hard or soft particles, and others.

The invention further provides a method for (simultaneous) delivery or for rendering available a combination of a lipophilic agent and a metal-based hydrophilic agent to a medium, the method comprising contacting the medium or administering to the medium or brining in contact with the medium an emulsion comprising a compound of the invention, the compound being provided in an interface formed between the lipophilic agent and an aqueous phase, wherein the compound is chemically associated with a metal of the metal-based hydrophilic agent; and causing dissociation of the metal of the metal-based hydrophilic agent to thereby deliver or render available to the medium both the lipophilic agent and the metal-based hydrophilic agent.

The medium may be a cell, a tissue, blood stream, an organ, a plant part, an organism, a solid or liquid formulation, a food product, etc.

The invention further provides a method for (simultaneous) delivering of a combination of a lipophilic pharmaceutical agent and a metal-based hydrophilic pharmaceutical agent to a cell, a tissue or a blood stream, the method comprising topically or systemically administering an emulsion comprising a compound of the invention, the compound being provided in an interface formed between the lipophilic pharmaceutical agent and an aqueous phase, wherein the compound is chemically associated with the metal-based hydrophilic pharmaceutical agent; and causing dissociation of the metal of the metal-based hydrophilic agent to thereby deliver or render available both the lipophilic agent and the metal-based hydrophilic agent to the cell tissue or blood stream.

In some embodiments, the compound is a peptide, as disclosed herein.

The delivery and release may take place in vivo, ex vivo or in vitro; the delivery and release may be to and in the blood stream, the GI track, the mouth, or any organ of the body; the delivery and release may be to and the skin, hair and nails. In other words, the emulsion may be administered topically, by injection (by e.g., parenteral, subcutaneous, intravenous, intramuscular, interperitoneal administration), orally, or by any other method of administration.

As disclosed and explained herein, upon dissociation of the metal-based hydrophilic agent, the amphiphilicity of the compound of the invention is lost, rendering the emulsion unstable, causing release of the lipophilic material being or contained in the oil phase of the emulsion. The release of the active materials renders them available to the tissue, thereby permitting their absorption by the tissue, diffusion into the tissue of blood vessels, association to the tissue etc. The dissociation of the metal-based hydrophilic agent may be triggered or may occur spontaneously under the conditions of use, delivery or in vivo conditions. The conditions causing or triggering dissociation of the metal-based hydrophilic agent include such may include pH (at a pH lower than pH6, the histidine groups are protonated and cannot bind the metal, thereby releasing the metal and causing release of the lipophilic agent as well), ionic strength (at very high ionic strengths, charge screening of the salt weakens the coordination between the metal and the metal-binding groups), presence or absence of better chelating agents, thermal conditions (an increase in temperature may weaken the bonds between the metal and the metal-binding groups due to thermal motion), and others.

The invention further provides a method for a (simultaneous) triggered release of a combination of metal-based hydrophilic agent and a lipophilic agent from an emulsion comprising a compound of the invention, wherein the compound has been provided in an interface formed between the lipophilic agent and an aqueous phase of the emulsion, and wherein the compound is chemically associated with a metal of the metal-based hydrophilic agent; and wherein following administration or contacting with a medium, applying to said medium at least one stimuli to cause dissociation of the metal of the metal-based hydrophilic agent to thereby release of both the lipophilic agent and the metal-based hydrophilic agent; said at least one stimulus being selected from pH, ionic strength, presence or absence of better chelating agents, thermal conditions, and others.

In some embodiments, the at least one stimulus occurs naturally or spontaneously in a cell, a tissue, an organ or a blood stream.

In some embodiments, the at least one stimulus is generated upon demand or is applied to the emulsion by externally affecting a change in the pH, ionic strength, presence or absence of better chelating agents, thermal conditions, etc., of the emulsion.

The invention further provides a kit comprising a surfactant of the invention, optionally provided in a solution or emulsion form; and instructions of use.

A further kit is provided, the kit comprising a first receptacle comprising a reversible surfactant according to the invention in a non-amphiphilic confirmation or form; and a second receptacle for emulsifying said reversible surfactant in presence of a metal ion or a compound comprising same; and instructions of use.

As used herein, the terms “moiety”, “functionality” and “group” are interchangeable.

The invention further provides:
A water-oil mixture comprising a lipophilic agent, a metal-based hydrophilic agent and a reversible surfactant comprising at least one metal-binding moiety and at least one hydrophobic functionality,
the reversible surfactant being switchable between an amphiphilic conformation and a non-amphiphilic conformation, whereby association between the at least one metal-binding moiety of the reversible surfactant and a metal ion of the metal-based hydrophilic agent induces emulsification of both the lipophilic agent and the metal-based hydrophilic agent,
wherein dissociation of the metal-based hydrophilic agent from the reversible surfactant triggers loss of amphiphilicity and release of the hydrophilic and lipophilic agents.

In some configurations of a mixture of the invention, the mixture is an emulsion wherein the at least one metal-binding moiety of the surfactant associates said metal-based hydrophilic agent at an interface between an aqueous phase comprising the metal-based hydrophilic agent and an oil phase comprising or being the lipophilic agent.

Also provided is an emulsion comprising a lipophilic agent, a metal-based hydrophilic agent and an amphiphilic surfactant comprising at least one metal-binding moiety and at least one hydrophobic functionality,
the at least one metal-binding moiety of the amphiphilic surfactant associating said metal-based hydrophilic agent at an interface between an aqueous phase comprising the metal-based hydrophilic agent and an oil phase comprising or being the lipophilic agent,
wherein dissociation of the metal-based hydrophilic agent from the amphiphilic surfactant triggers loss of amphiphilicity and release of the hydrophilic and lipophilic agents.

In some configurations of emulsions of the invention, the emulsions are water-in-oil (W/O), or oil-in-water (O/W), or a water-in-oil-in-water (W/O/W), or an oil-in-water-in-oil (O/W/O) emulsion.

In some configurations of emulsions of the invention, each of the metal-based hydrophilic agent and the lipophilic agent, independently, is selected amongst pharmaceutically active agents, cosmetically active agents, agriculturally active agents, food additives and dietary ingredients.

In some configurations of emulsions of the invention, the dissociation is triggered by a change in pH, a change in ionic strength, or a change in temperature.

In some configurations of emulsions of the invention, the surfactant is a linear or branched compound having an amphiphilic conformation and a non-amphiphilic conformation.

In some configurations of emulsions of the invention, the surfactant adopts an amphiphilic conformation in presence of a metal ion.

In some configurations of emulsions of the invention, the surfactant is a peptide.

In some configurations of emulsions of the invention, the peptide comprises two or more amino acids, one or more of the amino acids is a metal-binding amino acid and one or more of the other amino acids is a hydrophobic amino acid.

In some configurations of emulsions of the invention, the peptide is a dipeptide, a tripeptide, a tetrapeptide, a pentapeptide, a hexapeptide or a peptide having 7 or more amino acids.

In some configurations of emulsions of the invention, the peptide having 4, 5, 6 or 7 amino acids.

In some configurations of emulsions of the invention, the peptide comprises aromatic amino acids, optionally selected from histidine, tryptophan, tyrosine, naphthylalanine, and phenylalanine.

In some configurations of emulsions of the invention, the peptide comprises one or more metal-binding moieties capable of associating to a metal ion of monovalent, bivalent, trivalent and higher oxidation metals.

In some configurations of emulsions of the invention, the metal ion is selected from copper ions, calcium ions, chromium ions, magnesium ions, iron ions, zinc ions, platinum ions, molybdenum ions, technetium ions, vanadium ions, manganese ions, nickel ions, thallium ions, indium ions, silver ions, gold ions, cobalt ions, aluminum ions, ytterbium ions, europium ions, praseodymium ions, and gadolinium ions.

In some configurations of emulsions of the invention, the metal-binding moiety is capable of reversible associate to a metal ion.

In some configurations of emulsions of the invention, the metal-binding moiety is an atom or a group of atoms comprising an atom selected from sulfur, nitrogen, oxygen, and phosphorus.

In some configurations of emulsions of the invention, the surfactant is a peptide and wherein the metal-binding moiety is a nitrogen group of an amino acid.

In some configurations of emulsions of the invention, the amino acid is selected from histidine, arginine, proline and lysine.

In some configurations of emulsions of the invention, the amino acid is histidine.

In some configurations of emulsions of the invention, the surfactant is a peptide comprising 2 or more histidine amino acids.

In some configurations of emulsions of the invention, a histidine amino acid is provided at each peptide terminus, and wherein optionally at least one another histidine is provided along the peptide chain.

In some configurations of emulsions of the invention, the surfactant is a peptide comprising two or more histidine amino acids and one or more hydrophobic amino acid.

In some configurations of emulsions of the invention, the one or more hydrophobic functionality is a hydrocarbon having at least 6 carbon atoms, an aromatic group or a large chemical group having low or no solubility in water.

In some configurations of emulsions of the invention, the hydrophobic functionality is a hydrophobic amino acid.

In some configurations of emulsions of the invention, the hydrophobic amino acid is selected from glycine, alanine, valine, leucine, isoleucine, proline, phenylalanine, naphthylalanine, methionine, and tryptophan.

In some configurations of emulsions of the invention, the hydrophobic amino acid is tryptophan.

In some configurations of emulsions of the invention, the surfactant is a peptide comprising two or more histidine amino acids and two or more tryptophan amino acids.

In some configurations of emulsions of the invention, the surfactant is a tetrapeptide comprising two histidine amino acids and two tryptophan amino acids.

In some configurations of emulsions of the invention, the surfactant is a peptide selected from Trp-His-His-Trp (SEQ ID NO 1), Trp-His-Trp-His (SEQ ID NO 2), His-Trp-Trp-His (SEQ ID NO 3), and His-Trp-His-Trp (SEQ ID NO 4).

In some configurations of emulsions of the invention, the surfactant is a peptide, wherein the metal-binding moiety is histidine and the hydrophobic functionality is phenylalanine or naphthylalanine.

In some configurations of emulsions of the invention, the metal-based hydrophilic agent and the lipophilic agent are each selected from pharmaceutically acceptable drugs, medicaments or agents used in medicine.

In some configurations of emulsions of the invention, the pharmaceutically acceptable drugs, medicaments or agents are selected amongst cytotoxic drugs or chemotherapeutic drugs, anticancer drugs, anti-inflammatory drugs, antimicrobial drugs, antioxidants, antihistamines, hormones, analgesics, vitamins, anthelmintic, anti-arrhythmic drugs, anti-coagulants, anti-depressants, anti-diabetics, anti-diarrheal, anti-epileptics, anti-hypertensive agents, anti-malarial, anti-migraine agents, anti-muscarinic agents, immunosuppressants, anti-protozoal agents, anti-rheumatics, anti-thyroid agents, anxiolytics, sedatives, hypnotics and neuroleptics, antipsychotics, beta-blockers, cardiac inotropic agents, corticosteroids, cough suppressants, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents, oral vaccines, proteins, peptides and recombinant drugs, sex hormones and contraceptives, spermicides, stimulants, and combinations thereof.

In some configurations of emulsions of the invention, the metal-based hydrophilic agent and the lipophilic agent are each selected from cosmetically active agents.

In some configurations of emulsions of the invention, the cosmetically active agent is selected for inducing treatment or prevention of a skin disease or disorder, for improving skin condition or for affecting human skin, including hair and nails.

In some configurations of emulsions of the invention, the cosmetically active agent is selected amongst skin lightening agents, darkening agents, anti-acne agents, shine control agents, antimicrobial agents, anti-inflammatory agents, sunscreens, photo-protectors, antioxidants, keratolytic agents, nutrients, vitamins, energy enhancers, astringents, deodorants, hair growth enhancing agents, anticallous agents, fluorides, odor-control agents and combinations thereof.

In some configurations of emulsions of the invention, the metal-based hydrophilic agent and the lipophilic agent are each selected from agriculturally active agents suitable for application onto a plant part or a vicinity of a plant.

In some configurations of emulsions of the invention, the agriculturally active agent is selected from antimicrobial materials, insecticidal materials, insect-repelling materials, nutrients, fertilizers, pesticides, acaracides, herbicides, nemacides, rodenticides, entomopathogens, pheromones, attractants, plant growth regulators, plant hormones, insect growth regulators, chemosterilants, microbial pest control agents, phagostimulents, biological control agents, any combination of the aforementioned and combinations thereof.

In some configurations of emulsions of the invention, the metal-based hydrophilic agent and the lipophilic agent are each selected from food additives or dietary supplements, suitable for human or animal consumption.

In some configurations of emulsions of the invention, the food additive is selected from preservatives, coloring agents, flavoring agents, sweeteners, stabilizers, antioxidants, antimicrobial agents, nutrients, minerals, supplements, leavening agents, acids, acidity regulators, anticaking agents, antifoaming agents, bulking agents, color retention agents, glazing agents, humectants, thickeners and combinations thereof.

In some configurations of emulsions of the invention, the dietary supplement is selected from vitamins, flavonoids and bioflavonoids, carotenoids, phytosterols, polyphenols, carbohydrates, and proteins.

In some configurations of emulsions of the invention, the metal-based hydrophilic agent is a metal salt or a metal-ion containing agent.

In some configurations of emulsions of the invention, a metal ion of the metal salt or a metal ion of the metal-ion is or comprises copper, calcium, chromium, magnesium, iron, zinc, platinum, molybdenum, technetium, vanadium, manganese, nickel, thallium, indium, silver, gold, cobalt, aluminum, ytterbium, europium, praseodymium or gadolinium ions.

In some configurations of emulsions of the invention, the emulsion is for delivering nano or microparticles, wherein the nano or microparticles having surface groups enabling association with the metal binding moieties.

Also provided is a non-amphiphilic compound comprising one or more metal-binding moieties and one or more hydrophobic functionalities, said compound being reversibly convertible into an amphiphilic compound upon exposure to metal ions.

Further provided is a surfactant switchable between amphiphilic and non-amphiphilic conformations, the surfactant comprising one or more metal-binding moieties and one or more hydrophobic functionalities, wherein binding of a metal ion to the one or more metal-binding moieties triggers a reversible conformational change from the non-amphiphilic conformation to the amphiphilic conformation.

In some configurations of surfactants of the invention, the surfactant is for emulsifying both a lipophilic agent and a metal-based hydrophilic agent, said surfactant being switchable between amphiphilic and non-amphiphilic conformations, wherein in the amphiphilic conformation the surfactant establishes a chemical association between the metal-based hydrophilic agent and one or more metal binding moieties thereof, the surfactant further establishes an interaction between the lipophilic agent and one or more hydrophobic functionality thereof; the reversibility between the amphiphilic and non-amphiphilic conformations being metal-ion based.

In some configurations of surfactants of the invention, each of the metal-based hydrophilic agent and the lipophilic agent, independently, is selected amongst pharmaceutically active agents, cosmetically active agents, agriculturally active agents, food additives and dietary ingredients.

In some configurations of surfactants of the invention, the dissociation is triggered by a change in pH, a change in ionic strength, or a change in temperature.

In some configurations of surfactants of the invention, the surfactant is a linear or a branched compound having an amphiphilic conformation and a non-amphiphilic conformation.

In some configurations of surfactants of the invention, the surfactant is a peptide.

In some configurations of surfactants of the invention, the peptide comprises two or more amino acids, one or more of the amino acids is a metal-binding amino acid and one or more of the other amino acids is a hydrophobic amino acid.

In some configurations of surfactants of the invention, the peptide is a dipeptide, a tripeptide, a tetrapeptide, a pentapeptide, a hexapeptide or a peptide having 7 or more amino acids.

In some configurations of surfactants of the invention, the peptide having 4, 5, 6 or 7 amino acids.

In some configurations of surfactants of the invention, the peptide comprises aromatic amino acids, optionally selected from histidine, tryptophan, tyrosine, naphthylalanine, and phenylalanine.

In some configurations of surfactants of the invention, the peptide comprises one or more metal-binding moieties capable of associating to a metal ion of monovalent, bivalent, trivalent and higher oxidation metals.

In some configurations of surfactants of the invention, the metal ion is selected from copper ions, calcium ions, chromium ions, magnesium ions, iron ions, zinc ions, platinum ions, molybdenum ions, technetium ions, vanadium ions, manganese ions, nickel ions, thallium ions, indium ions, silver ions, gold ions, cobalt ions, aluminum ions, ytterbium ions, europium ions, praseodymium ions, and gadolinium ions.

In some configurations of surfactants of the invention, the metal-binding moiety is capable of reversible associate to a metal ion.

In some configurations of surfactants of the invention, the metal-binding moiety is an atom or a group of atoms comprising an atom selected from sulfur, nitrogen, oxygen, and phosphorus.

In some configurations of surfactants of the invention, the surfactant is a peptide and wherein the metal-binding moiety is a nitrogen group of an amino acid.

In some configurations of surfactants of the invention, the amino acid is selected from histidine, arginine, proline and lysine.

In some configurations of surfactants of the invention, the amino acid is histidine.

In some configurations of surfactants of the invention, the surfactant is a peptide comprising 2 or more histidine units.

In some configurations of surfactants of the invention, a histidine unit is provided at each peptide termini, and wherein optionally at least one another histidine is provided along the peptide chain.

In some configurations of surfactants of the invention, the surfactant is a peptide comprising two or more histidine binding moieties and one or more hydrophobic functionalities.

In some configurations of surfactants of the invention, the one or more hydrophobic functionality is a hydrocarbon having at least 6 carbon atoms, an aromatic group or a large chemical group having low or no solubility in water.

In some configurations of surfactants of the invention, the hydrophobic functionality is a hydrophobic amino acid.

In some configurations of surfactants of the invention, the hydrophobic amino acid is selected from glycine, alanine, valine, leucine, isoleucine, proline, phenylalanine, methionine, and tryptophan.

In some configurations of surfactants of the invention, the hydrophobic amino acid is tryptophan.

In some configurations of surfactants of the invention, the surfactant is a peptide comprising two or more histidine units and two or more tryptophan units.

In some configurations of surfactants of the invention, the surfactant is a tetrapeptide comprising two histidine units and two tryptophan units.

In some configurations of surfactants of the invention, the surfactant is a peptide selected from Trp-His-His-Trp (SEQ ID NO 1), Trp-His-Trp-His (SEQ ID NO 2), His-Trp-Trp-His (SEQ ID NO 3), and His-Trp-His-Trp (SEQ ID NO 4).

In some configurations of surfactants of the invention, the surfactant is an amino acid, wherein the metal-binding moiety is histidine and the hydrophobic functionality is phenylalanine or naphthylalanine.

In some configurations of surfactants of the invention, the metal-based hydrophilic agent and the lipophilic agent are each selected from pharmaceutically acceptable drugs, medicaments or agents used in medicine.

In some configurations of surfactants of the invention, the pharmaceutically acceptable drugs, medicaments or agents are selected amongst cytotoxic drugs or chemotherapeutic drugs, anticancer drugs, anti-inflammatory drugs, antimicrobial drugs, antioxidants, antihistamines, hormones, analgesics, vitamins, anthelmintic, anti-arrhythmic drugs, anti-coagulants, anti-depressants, anti-diabetics, anti-diarrheal, anti-epileptics, anti-hypertensive agents, anti-malarial, anti-migraine agents, anti-muscarinic agents, immunosuppressants, anti-protozoal agents, anti-rheumatics, anti-thyroid agents, anxiolytics, sedatives, hypnotics and neuroleptics, antipsychotics, beta-blockers, cardiac inotropic agents, corticosteroids, cough suppressants, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents, oral vaccines, proteins, peptides and recombinant drugs, sex hormones and contraceptives, spermicides, stimulants, and combinations thereof.

In some configurations of surfactants of the invention, the metal-based hydrophilic agent and the lipophilic agent are each selected from cosmetically active agents.

In some configurations of surfactants of the invention, the cosmetically active agent is selected for inducing treatment or prevention of a skin disease or disorder, for improving skin condition or for affecting human skin, including hair and nails.

In some configurations of surfactants of the invention, the cosmetically active agent is selected amongst skin lightening agents, darkening agents, anti-acne agents, shine control agents, antimicrobial agents, anti-inflammatory agents, sunscreens, photo-protectors, antioxidants, keratolytic agents, nutrients, vitamins, energy enhancers, astringents, deodorants, hair growth enhancing agents, anticallous agents, fluorides, odor-control agents and combinations thereof.

In some configurations of surfactants of the invention, the metal-based hydrophilic agent and the lipophilic agent are each selected from agriculturally active agents suitable for application onto a plant part or a vicinity of a plant.

In some configurations of surfactants of the invention, the agriculturally active agent is selected from antimicrobial materials, insecticidal materials, insect-repelling materials, nutrients, fertilizers, pesticides, acaracides, herbicides, nemacides, rodenticides, entomopathogens, pheromones, attractants, plant growth regulators, plant hormones, insect growth regulators, chemosterilants, microbial pest control agents, phagostimulents, biological control agents, any combination of the aforementioned and combinations thereof.

In some configurations of surfactants of the invention, the metal-based hydrophilic agent and the lipophilic agent are each selected from food additives or dietary supplements, suitable for human or animal consumption.

In some configurations of surfactants of the invention, the food additive is selected from preservatives, coloring agents, flavoring agents, sweeteners, stabilizers, antioxidants, antimicrobial agents, nutrients, minerals, supplements, leavening agents, acids, acidity regulators, anticaking agents, antifoaming agents, bulking agents, color retention agents, glazing agents, humectants, thickeners and combinations thereof.

In some configurations of surfactants of the invention, the dietary supplement is selected from vitamins, flavonoids and bioflavonoids, carotenoids, phytosterols, polyphenols, carbohydrates, and proteins.

In some configurations of surfactants of the invention, the metal-based hydrophilic agent is a metal salt or a metal-ion containing agent.

In some configurations of surfactants of the invention, a metal ion of the metal salt or a metal ion of the metal-ion containing agent is or comprises copper, calcium, chromium, magnesium, iron, zinc, platinum, molybdenum, technetium, vanadium, manganese, nickel, thallium, indium, silver, gold, cobalt, aluminum, ytterbium, europium, praseodymium or gadolinium ions.

In some configurations of surfactants of the invention, the surfactant is for delivering nano or microparticles, wherein the nano or microparticles having surface groups enabling association with the metal binding moieties.

Also provided is a method for (simultaneous) delivery or for rendering available a combination of a lipophilic agent and a metal-based hydrophilic agent to a medium, the method comprising
contacting the medium or administering to the medium or bringing in contact with the medium an emulsion comprising a surfactant according to the invention, the surfactant being provided in an interface formed between the lipophilic agent and an aqueous phase, wherein the surfactant is chemically associated with a metal of the metal-based hydrophilic agent; and
causing dissociation of the metal of the metal-based hydrophilic agent to thereby deliver or render available to the medium both the lipophilic agent and the metal-based hydrophilic agent.

In some configurations of methods of the invention, the surfactant is a peptide.

In some configurations of all mixtures, emulsions, surfactants, methods and other aspects of the invention, the surfactant is a peptide selected from Trp-His-His-Trp (SEQ ID NO 1), Trp-His-Trp-His (SEQ ID NO 2), His-Trp-Trp-His (SEQ ID NO 3), and His-Trp-His-Trp (SEQ ID NO 4).

In some configurations of all mixtures, emulsions, surfactants, methods and other aspects of the invention, the surfactant is Trp-His-His-Trp (SEQ ID NO 1).

In some configurations of all mixtures, emulsions, surfactants, methods and other aspects of the invention, the surfactant is Trp-His-Trp-His (SEQ ID NO 2).

In some configurations of all mixtures, emulsions, surfactants, methods and other aspects of the invention, the surfactant is His-Trp-Trp-His (SEQ ID NO 3).

In some configurations of all mixtures, emulsions, surfactants, methods and other aspects of the invention, the surfactant is His-Trp-His-Trp (SEQ ID NO 4).

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D. Peptide-stabilized Emulsion. (A) A scheme illustrating the formation of stable emulsions. Upon binding certain metal ions through the histidines, the peptide forms an amphiphilic complex with a polar head and two hydrophobic tails of the tryptophans. When this complex is mixed with a non-aqueous phase, it can stabilize emulsion droplets. (B) Mixture of chloroform and water (i) with the peptide (ii), Cu²⁺ ions (iii), and the peptide alongside with Cu²⁺ ions (iv). The emulsion is formed only when both the peptide and the Cu²⁺ ions are present. (C) Oil-in-water emulsion stabilized with a peptide-Zn²⁺ complex using castor oil labeled with Nile red. (D) Paraffin oil-water emulsion with FITC labeling for the peptide.

FIGS. 2A-C. Emulsion characterization. (A) A w/o emulsion formed using 90% (v/v) castor oil labeled with Nile red. (B) Excitation and emission spectra for the peptide (left) and a fluorescence microscopy image of the peptide fibrous structures. (C) Images depicting the reversible assembly and disassembly of the emulsion through pH changes.

FIGS. 3A-E. Emulsion stability. (A) Stability of chloroform in water emulsions. The left vial in each image is the peptide-Zn²⁺-stabilized emulsion and the right vial is the SDS-stabilized emulsion. (B) A peptide-Zn²⁺-stabilized chloroform in water emulsion after addition of 1 equiv EDTA. (C) Chloroform-water mixtures with 1 mM peptide, 1 mM Zn²⁺, or increasing concentrations of the peptide-Zn²⁺ complex. (D) Peptide-Zn²⁺-stabilized chloroform in water emulsions with different oil-water ratios. (E) Thermal stability of chloroform in water emulsions. The left vial in each image is the peptide-Zn²⁺-stabilized emulsion and the right vial is the SDS-stabilized emulsion.

FIGS. 4A-B. Emulsions with different metal ions. (A) Chloroform in water mixtures with the peptide (1 mM) and different metal ions (1 mM). Arrows point to samples with metal ions that formed stable emulsions with the peptide (B) Light and fluorescent microscopy images of the peptide-Eu³⁺-stabilized emulsion.

FIGS. 5A-B. Termini-capped peptides. (A) MALDI TOF/TOF analysis for NH₂-Trp-His-His-Trp-CONH₂ (SEQ ID NO 5, left) and for AcN-Trp-His-His-Trp-CONH₂ (SEQ ID NO 6, right). (B) Chloroform in water mixtures with the AcN-Trp-His-His-Trp-CONH₂ (SEQ ID NO 6) peptide (1 mM) and different metal ions (1 mM). Arrows point to samples with metal ions that formed stable emulsions with the peptide.

FIG. 6. Benesi-Hildebrand plot. The stability constant derived from this plot was 6918.3±0.2 M⁻¹, similar to the known value for imidazole.

FIGS. 7A-C: Spectroscopic characterization of the peptide-metal complexation. (A) UV-vis titration of the peptide with Cu²⁺ ions. (B) A Job's plot for the peptide-Cu²⁺ complex. The original measurements are displayed as circles, and the measurements after subtraction of the absorbance of the excess Cu²⁺ are displayed as triangles. (C) The CD spectra for the free peptide, peptide with Fe²⁺ ions, and peptide with Cu²⁺ or Zn²⁺ ions.

FIG. 8. Control peptides. Chloroform-water mixtures with 1 mM of the peptide derivatives and 1 mM Cu²⁺ ions.

FIG. 9. Emulsion pH-dependent stability. Representative light microscopy images of emulsions stabilized with a peptide-Zn²⁺ complex using paraffin oil, after addition to buffers with varying pH values. Scale bar, 50 μm.

FIGS. 10A-E: NMR analysis. (A) The fingerprint region of the peptide spectrum during titration with Zn²⁺ ions and with CDCl₃ (top spectrum). (B-D) Low energy, 10-member ensembles of the free peptide (B), the peptide bound to Zn²⁺ (C), and low energy, 8-member ensemble of the peptide bound to Zn²⁺ in the emulsion (D). (E) A hypothetical assembly mode of the peptide bound to Zn²⁺ in the emulsion.

FIGS. 11A-C: The peptide-metal emulsion as a drug delivery system. (A) Emulsion stability based on the change of the turbidity of emulsions stabilized with a peptide-Zn²⁺ complex using paraffin oil at three pH values. (B) Cumulative release of PTX from peptide-stabilized emulsions at two pH values. (C) Cell viability results with HeLa cells for PTX dissolved in DMSO (triangles), for the empty emulsion (circles), and for the PTX-encapsulated emulsion (squares). The data points and error bars represent the arithmetic mean and standard deviation values of triplicate measurements.

FIGS. 12A-H. ζ potential measurements. (A) DLS size distribution for the peptide-Zn²⁺-stabilized emulsion. (B) DLS size distribution for the peptide-Zn²⁺-stabilized emulsion after filtration with a 0.45 μm filter. (C) DLS size distribution for the peptide-Zn²⁺-stabilized emulsion immediately after preparation. (D) DLS size distribution for the same emulsion after 35 days. (E) ζ-potential distribution for a peptide-Zn²⁺-stabilized emulsion. (F) ζ-potential distribution for the same emulsion with ~0.2 equiv of H₆—Rh. (G) ζ-potential distribution for a peptide-Cu²⁺-stabilized emulsion. (H) ζ-potential distribution for the same emulsion with 0.2 μg/mL of the HRP probe.

FIG. 13. Emulsion Functionalization. Emulsion decorated with the H₆-FITC peptide 15 min after preparation (left) and the agglomerated emulsion 21.4 h after preparation (right).

FIGS. 14A-E: Emulsion functionalization. (A and B) Peptide-stabilized emulsion (left) and SDS-stabilized emulsion (right) with H6-Rh (A) and with the HRP-luminol system (B). A fluorescence intensity line scan (white line in the images) is displayed beneath the microscope images. (C) A scheme presenting the decoration of the peptide-stabilized emulsion against the SDS-stabilized emulsion. (D) The stability of the functionalization with H6-FITC, based on the change in the fluorescence intensity at the edge of the droplets relative to the fluorescence in the aqueous phase. The data points and error bars represent the arithmetic mean and standard deviation values of triplicate measurements. (E) Images of an emulsion containing Nile red and decorated with H6-FITC at pH 7.5 (left), 6.2 (middle), and 4.8 (right). A red and green fluorescence intensity line scan (white line in the images, green fluorescence as a straight line, red fluorescence as a dotted line) is displayed beneath the microscope images.

FIGS. 15A-B. Peptide identity and purity. (A) Analytical HPLC chromatogram (280 nm) and MS analysis for WHHW (SEQ ID NO 1). (B) MALDI TOF/TOF analysis for the H₆—Rh (left) and the H₆-FITC (right) peptides.

FIG. 16. The full ¹H-NMR spectrum of the free peptide with assigned peaks in zoomed images.

FIGS. 17A-C. NMR analysis. (A) Hε₁ peaks of the tryptophan side chains during the titration with Zn²⁺ ions and with CDCl₃. (B) The aliphatic region of the peptide during titration with Zn²⁺ ions and with CDCl₃. (C) The aromatic region of the peptide during titration with Zn²⁺ ions and with CDCl₃ by 2D COSY NMR. The lines represent 0 mM Zn²⁺, 0.6 mM, 1.2 mM, 1.8 mM, 2.4 mM, 3 mM, 6 mM, and an addition of 15% (v/v) CDCl₃, from bottom to top.

FIGS. 18A-C. Emulsion NMR analysis. (A) ¹H-¹H-NMR Fingerprint region of the peptide with ROESY, TOCSY, and COSY peaks. (B) 10-member ensemble of the peptide bound to Zn²⁺ without coordination to the metal ion in the structure calculation (RMSD values: Backbone 0.90 Å, heavy atoms 1.83 Å, heavy atoms of His residues 1.39 Å). (C) A hypothetical assembly mode of the peptides at the emulsion interface, allowing salt bridge interactions between layers of peptides.

FIG. 19. Affinity of different metal ions. The basis set superposition error (BSSE) corrected binding energy calculated for the different metal ions to bind the histidines (HH), tryptophan-histidine (WH), or the tryptophans (WW), the latter of which included C-terminus involvement. The lowest energy refers to the most strongly bound conformation, none of which are the WH conformation. Inset are the HH and WW conformations of Cu²⁺.

FIGS. 20A-B. (A) The peptide (10 mM) forms fibrous structures after 24 h in water. (B) When the peptide (5 mM) is mixed with Cu²⁺ ions (5 mM), it forms spherical aggregates alongside the fibrous structures.

### DETAILED DESCRIPTION OF EMBODIMENTS

### Complexation-Triggered Amphiphilicity

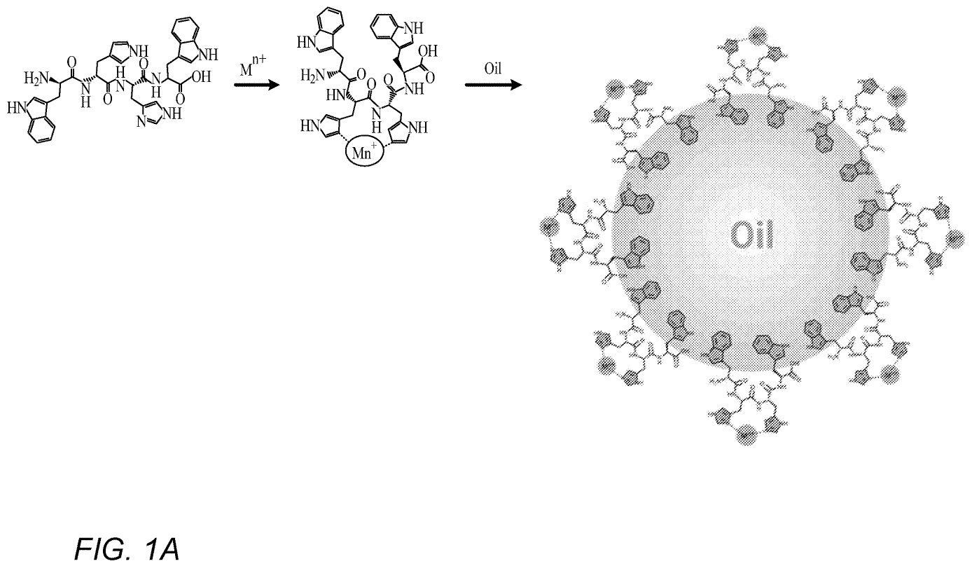

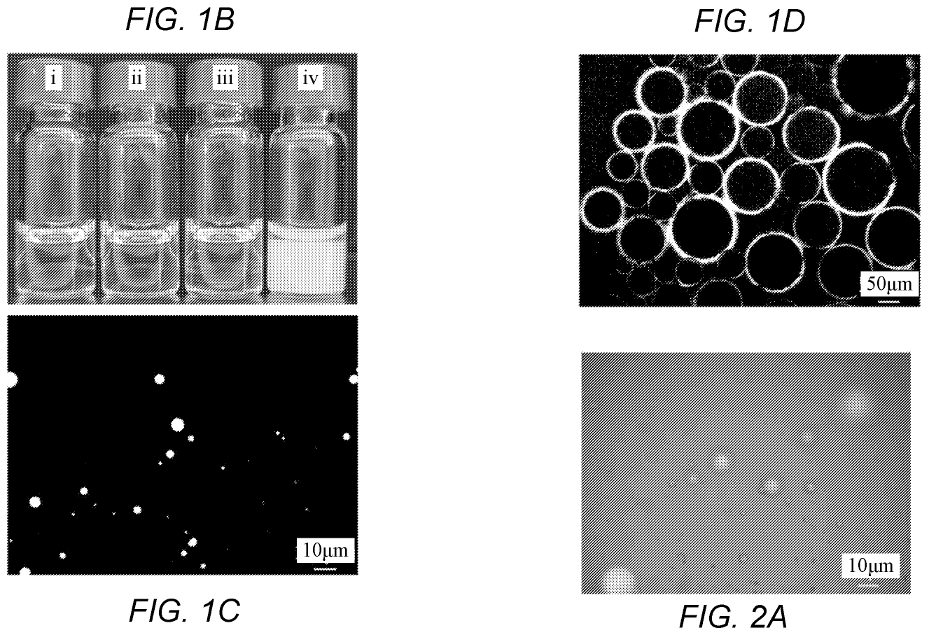

We designed the peptide Trp-His-His-Trp (WHHW, SEQ ID NO 1), which contains a metal-binding dihistidine moiety surrounded by two hydrophobic amino acids. When an aqueous solution of the peptide and certain metal ions are mixed with a non-aqueous phase, an emulsion is formed (FIG. 1B). This occurs only in presence of both the peptide and the metal ions, indicating that this emulsion is stabilized by the formation of a peptide-metal complex. Depending on the ratio between the aqueous and non-aqueous phases, o/w or w/o emulsions can be formed (FIG. 1C). The peptide has weak inherent fluorescence, but its presence on the interface of the emulsion droplets was inferred by labeling it with fluorescein isothiocyanate (FITC) (FIG. 1D).

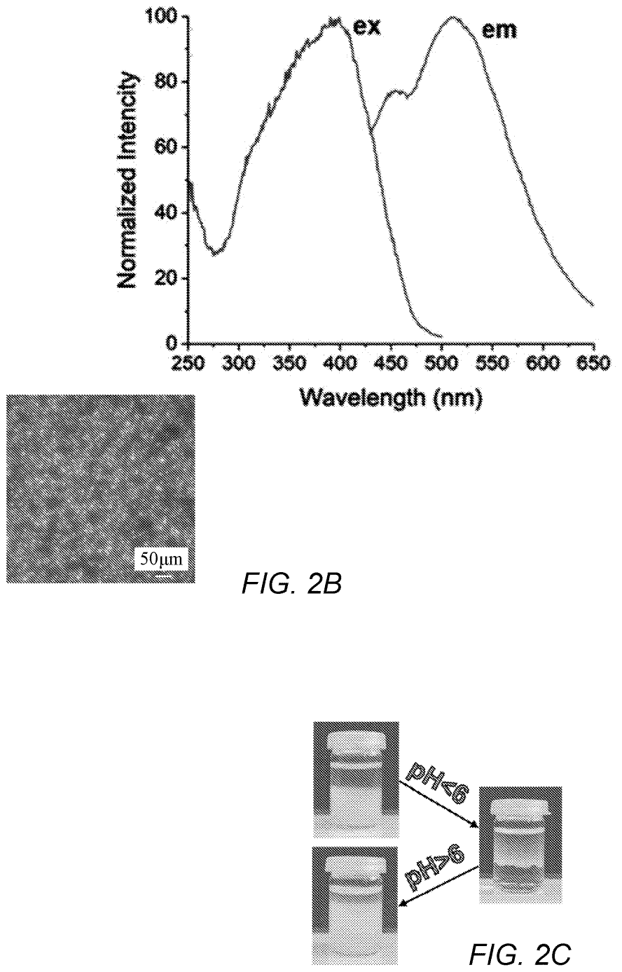

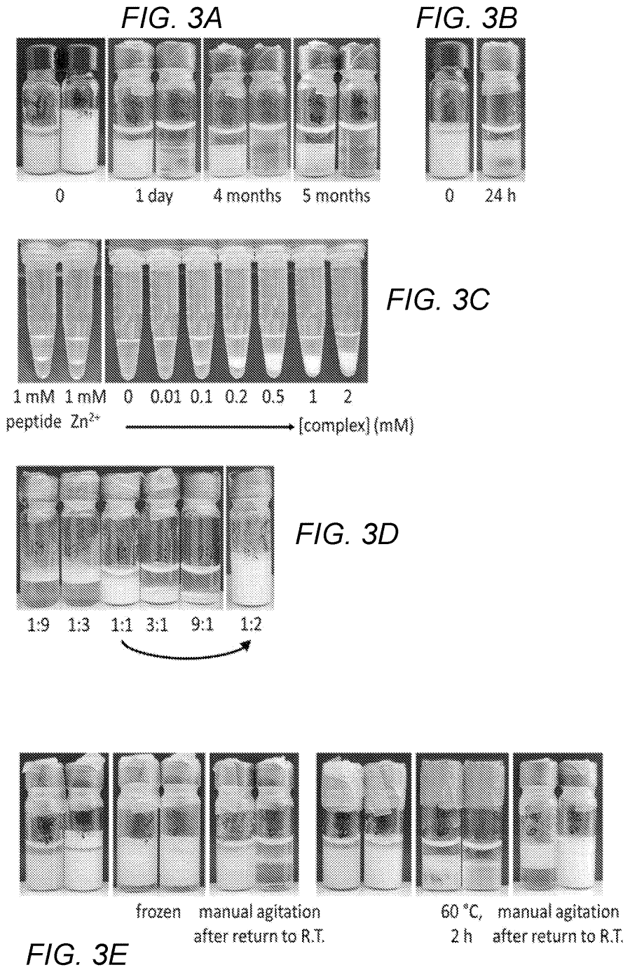

Since the stability of the emulsion depends on the stability of the complex, it can be reversibly disassembled by lowering the pH, thus protonating the imidazole groups of the histidines and preventing their coordination to the metal ions (FIG. 2C). By increasing the pH again, the emulsion can be reassembled. With chloroform as the non-aqueous phase, the emulsion has a critical micelle concentration of ~0.2 mM and it is stable for at least 4 months at room temperature (FIG. 3A). Adding ethylenediaminetetraacetic acid (EDTA) to remove the metal ions visibly disassembled the emulsion after ~24 h (FIG. 3B).

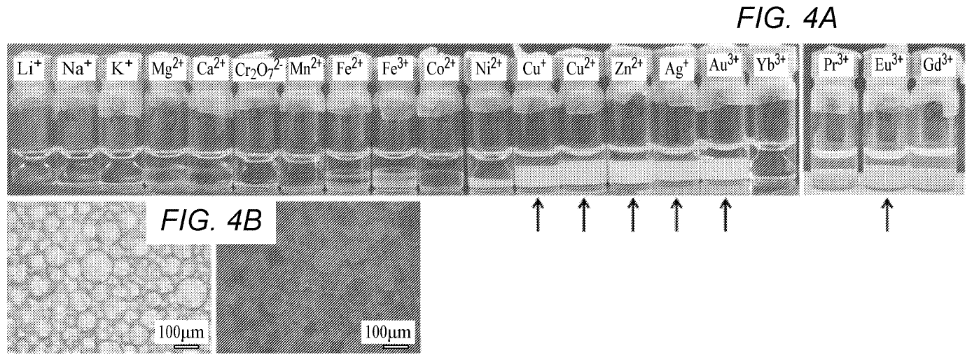

The metal ions that favor the formation of stable emulsions are Cu⁺, Cu²⁺, Zn²⁺, Ag⁺, and Au³⁺ (FIG. 4A). The reason for this selectivity may be that metal ions that are harder in nature (according to the ‘hard and soft acids and bases’ theory) such as Ni²⁺, Co²⁺, or Fe²⁺, have a stronger affinity to additional harder oxygen-based ligands, such as the peptide C-terminus. The same trend was observed in density-functional theory (DFT) simulations (FIG. 19). In these simulations Cu⁺, Cu²⁺, Zn²⁺, Ag⁺, and Au³⁺ preferred to bind the histidines, whereas Ni²⁺, Co²⁺, and Fe²⁺ preferred the termini of the peptide. By interacting with one of the aromatic amino acids at the termini, these ions will form complexes that do not allow folding into an amphiphilic complex. Stable emulsions were also prepared using Eu³⁺ ions. These emulsion droplets showed stronger fluorescence, possibly derived from the incorporation of Eu³⁺ in the complex (FIG. 4B).

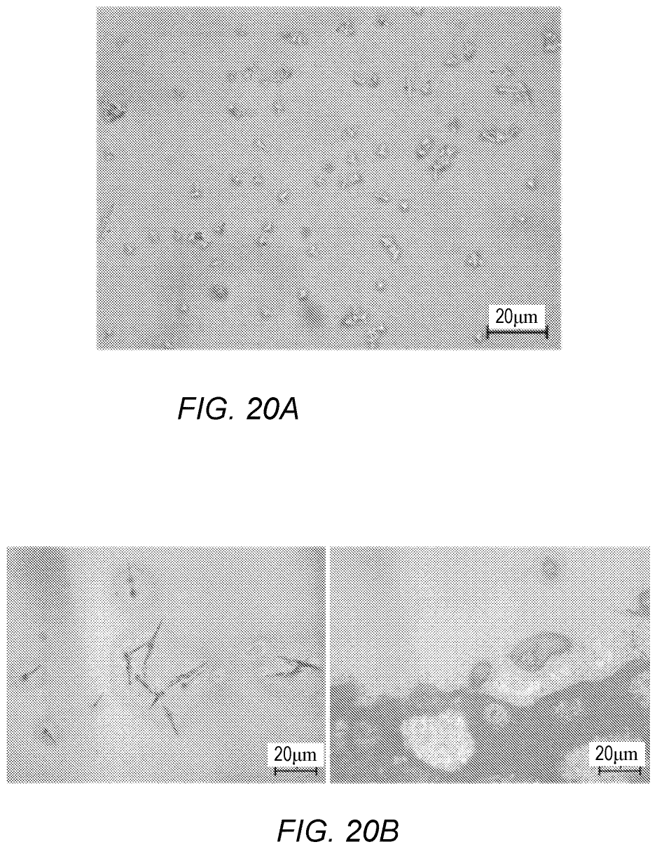

Using coarse-grained molecular dynamics (CGMD), we simulated the investigated peptide in an aqueous environment, in the presence of divalent cations, and in the presence of divalent cations as well as octane molecules. Two derivatives of the peptide were also simulated: Trp-Ala-Ala-Trp (WAAW, SEQ ID NO 7) and Ala-His-His-Ala (AHHA, SEQ ID NO 8). These peptides were investigated to reveal the significance of the metal binding moiety and of the hydrophobic amino acids. Our results show that WHHW (SEQ ID NO 1) self-assembles into a fibrous structure in water, a spherical structure in the presence of ions, and it forms micelles with the hydrophobic phase (FIG. 2). These results agree with experimental observations of fibrous peptide structures in water, and spherical aggregates in the presence of Cu²⁺ ions (FIGS. 20A-B). In the case of AHHA (SEQ ID NO 8), only a weak trend towards assembly in water was observed and an emulsion was not formed with octane. This may occur due to the lack of large hydrophobic groups that increase the peptide's amphiphilicity. In the case of WAAW (SEQ ID NO 7), the fibrous/spherical self-assembly relationship with the ions was reversed and only a loose emulsion was formed, whereby much of the hydrophobic phase was exposed to the water. The lack of a metal-binding moiety that can act as a lock, holding the peptide in an amphiphilic conformation, may cause this destabilization of the emulsion droplets.

### Spectroscopic Study of the Complex

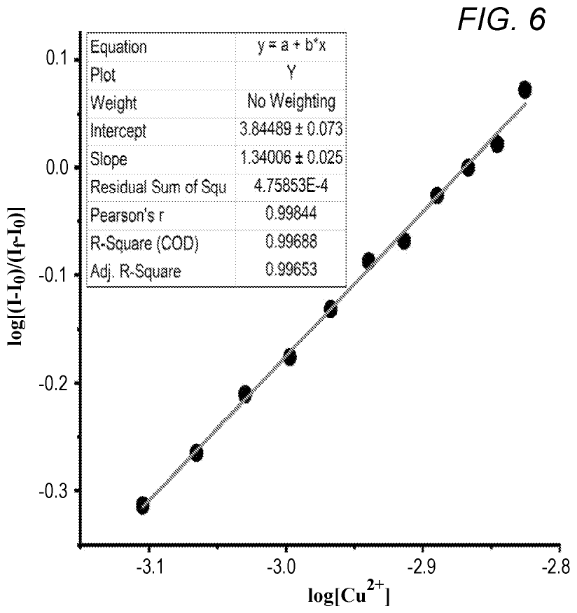

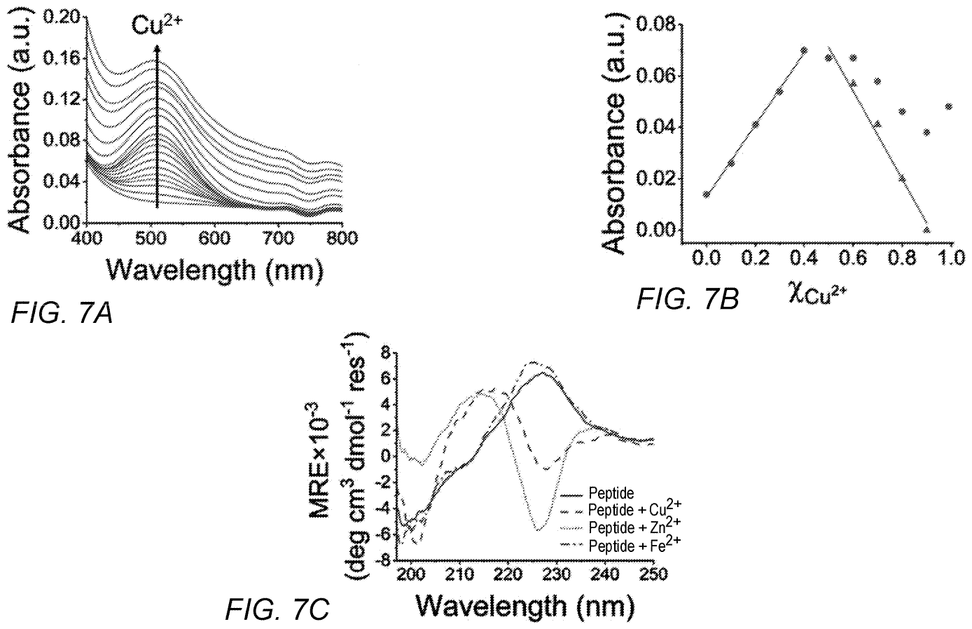

The peptide-Cu²⁺ complex displayed a red color with an absorbance peak at ~510 nm (FIG. 7A) that increased with the addition of the metal ions up to 1 eq. (FIG. 3A). A Benesi-Hildebrand plot showed a good linear fit, indicating a 1:1 binding ratio between the peptide and the metal (FIG. 6). A Job's plot was prepared as well with the peptide and Cu²⁺ ions (FIG. 7B). In the samples with excess Cu²⁺ ions (those above χ=0.5) the absorbance increased without an increase in the complex concentration. This may be because of the formation of Cu(OH)₂ precipitates from the excess Cu²⁺ ions due to the alkaline pH. To negate this effect, the absorbance values of solutions that contained the concentration of the excess Cu²⁺ were subtracted from the original measurements. This yielded good linear fittings that indicate a 1:1 binding ratio.

The conformational change of the peptide's structure upon addition of Cu²⁺ or Zn²⁺ ions can be inferred from circular dichroism (CD) measurements. The spectrum of a free peptide indicated a random coil conformation, due to the negative peak around 200 nm (FIG. 7C). The same conformation was obtained for the peptide in the presence of Fe²⁺ ions, which could not form an emulsion. In contrast, when the peptide was present with Cu²⁺ or Zn²⁺ ions, the CD spectra indicated a β-turn structure with peaks around 225 nm. Interestingly, the higher intensity of this peak in the spectrum of the peptide with Zn²⁺ ions, suggests that Zn²⁺ ions are more effective than Cu²⁺ ions in forming the B-turn structure. This further supports the ability of these metal ions to fold the peptide into an amphiphilic complex by introducing a turn to the peptide's structure, bringing the hydrophobic groups closer together.

To investigate the parts of the peptide that are necessary for the formation of the emulsion, we mixed different derivatives of the peptide with Cu²⁺ ions. The same absorbance peak at ~510 nm was also observed for the other dihistidine-containing tetrapeptides: Phe-His-His-Phe (FHHF, SEQ ID NO 9), Tyr-His-His-Tyr (YHHY, SEQ ID NO 10), and AHHA (SEQ ID NO 8), suggesting the formation of similar coordination of the metal through the histidines. Under the basic pH conditions of the experiment, the solution of Cu²⁺ ions formed Cu(OH)₂ precipitates that have an absorbance peak at ~650 nm. A similar spectrum was obtained for WAAW (SEQ ID NO 7) and for a single tryptophan amino acid, indicating the necessity of histidine for forming the complex. The mixtures of Cu²⁺ with a single histidine as well as with dihistidine show a peak around 590 nm as the metal ions interact with the imidazole moieties. Lastly, the peptide Trp-His (WH), displayed an absorbance peak similar to that of the Cu(OH)₂, with a shift towards that of the histidine and dihistidine peaks.

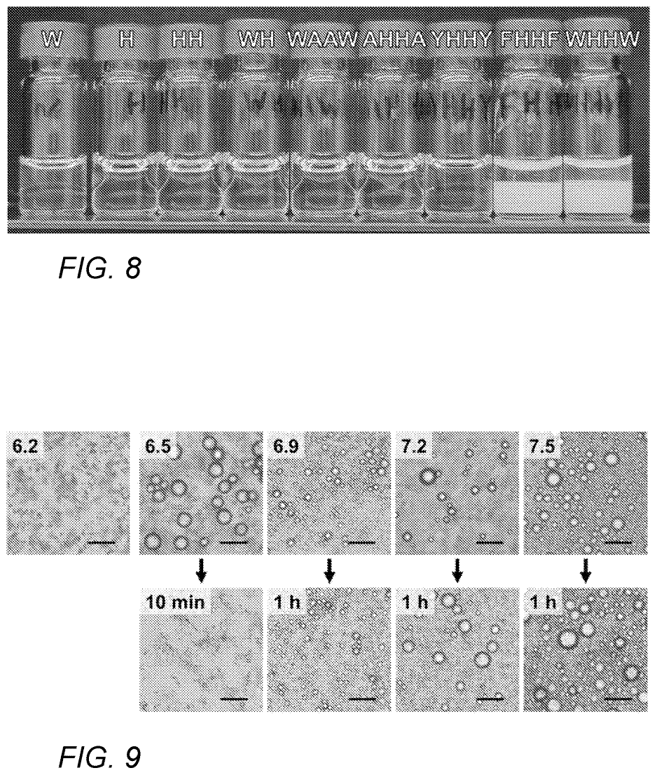

The same peptide derivatives in the presence of Cu²⁺ ions, were further mixed with chloroform and manually agitated to form emulsions (FIG. 8). The only peptide that formed emulsions other than WHHW (SEQ ID NO 1) was FHHF (SEQ ID NO 9), albeit the emulsion was less stable. This suggests that to create stable emulsions, the termini amino acids have to be both hydrophobic and bulky, thus decreasing the distance between them in the amphiphilic complex.

### Structural Analysis

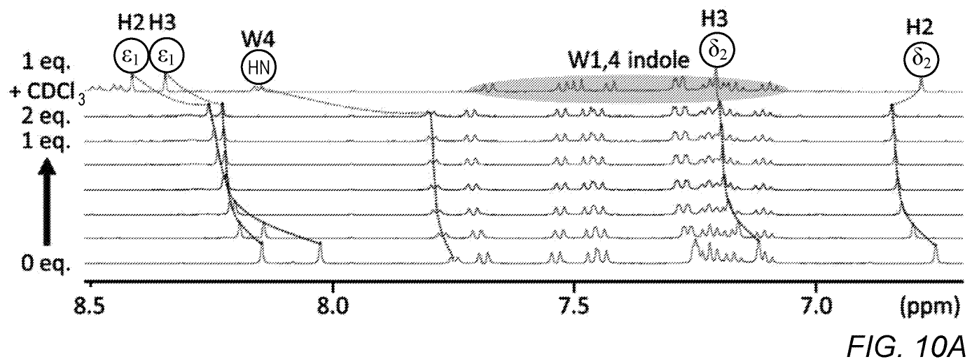

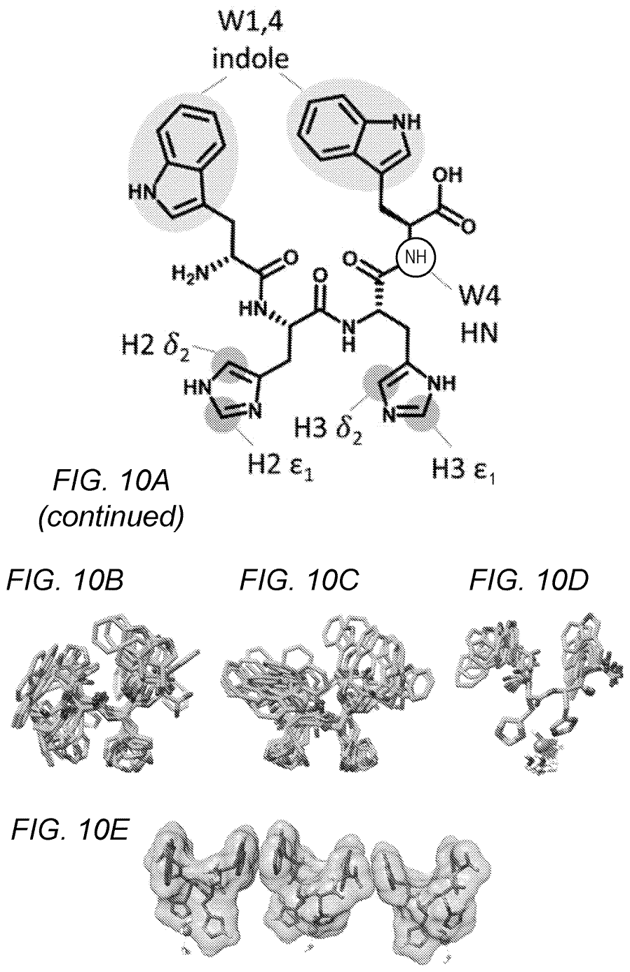

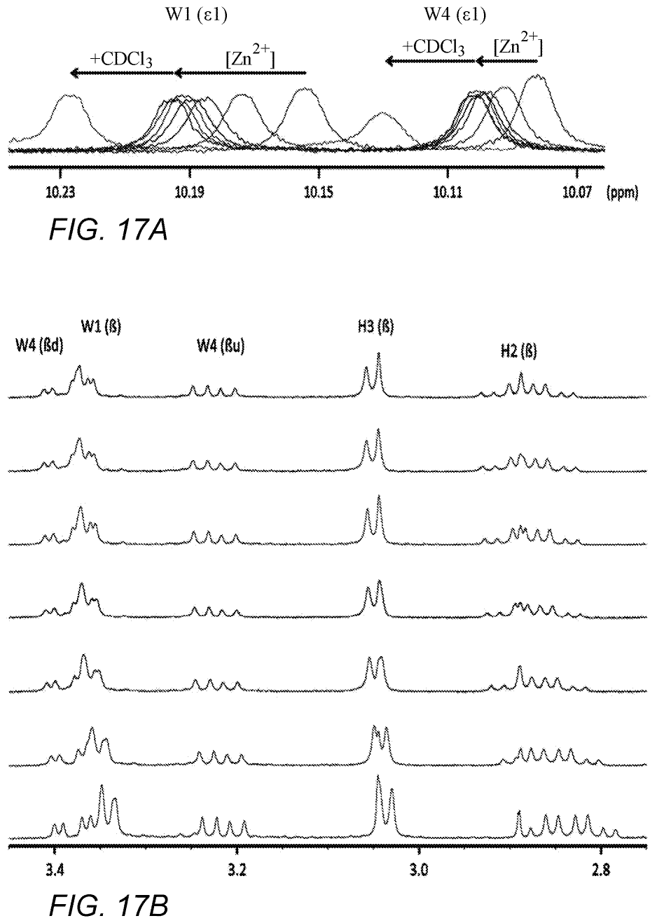

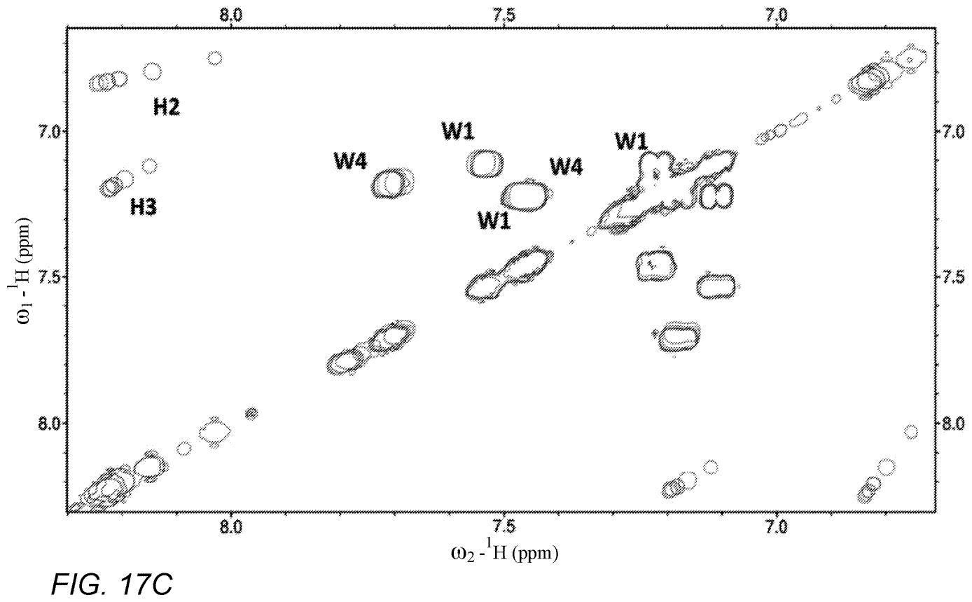

To further investigate the structural changes of the peptide, we performed 1D and 2D ¹H-NMR measurements. Titrating the peptide with Zn²⁺ ions corroborated that the metal binds through the imidazole groups, as shown from the difference between the values of the chemical shift (Δδₐ) of Hδ₂ and Hε₁ of both histidines before and after binding the metal ions (FIG. 10A and Table 1). Meanwhile, the tryptophan groups displayed little to no chemical shift changes during the titration. Adding more than 1 eq. of Zn²⁺ ions to the peptide did not cause further chemical shifts, as expected from the previous spectroscopic results. However, after adding ~15% (v/v) of CDCl₃, most of the polar groups displayed significant changes as indicated from the Δδ_{b} values (δₑₘᵤₗₛᵢₒₙ−δ_{bound}). These included the Hε₁ groups of both histidines, the HN group of the N-terminus, and the Hε₁ groups of both tryptophans (FIG. 17A). These results point to a significant rearrangement process that the peptide undergoes upon its repositioning at the water-chloroform interface. In this process, the polar groups will be the ones that are most repulsed by the non-aqueous medium, especially the N-terminal amine, which is adjacent to the hydrophobic indole group of the first tryptophan.

TABLE 1
NMR analysis. ¹H chemical shift values for the free
peptide, peptide with Zn²⁺ ions (1 eq.), and peptide
with Zn²⁺ ions and with 15% (v/v) CDCl₃.

| | | Apo | Zn²⁺ | CDCl₃ | Δδa | Δδb |
|---|---|---|---|---|---|---|
| Residue | Proton | ppm | ppm | ppm | (Zn²⁺-Apo) | (CDCl₃—Zn²⁺) |
| W1 | δ₁ | 7.25 | 7.29 | 7.29 | 18.22 | 0.05 |
| | ε₁ | 10.15 | 10.19 | 10.23 | 20.0 | 17.1 |
| | ε₃ | 7.47 | 7.47 | 7.49 | −0.4 | 11.4 |
| | ζ₂ | 7.54 | 7.53 | 7.52 | −5.1 | −2.1 |
| | ζ₃ | 7.20 | 7.20 | 7.22 | −0.4 | 7.7 |
| | η₂ | 7.10 | 7.11 | 7.10 | 2.5 | −7.3 |
| H2 | HN | n.d. | n.d. | 8.488 | n.d. | n.d. |
| | H_{β} | 2.83 | 2.88 | 2.872 | 25.7 | −3.6 |
| | δ₂ | 6.75 | 6.84 | 6.78 | 43.8 | −29.0 |
| | ε₁ | 8.03 | 8.24 | 8.41 | 109.136 | 85.363 |
| H3 | HN | n.d. | n.d. | 8.445 | n.d. | n.d. |
| | H_{β} | 3.03 | 3.05 | 3.05 | 8.5 | 0.5 |
| | δ₂ | 7.19 | 7.19 | 7.21 | 37.8 | 6.1 |
| | ε₁ | 8.15 | 8.23 | 8.35 | 39.6 | 60.6 |
| W4 | HN | 7.75 | 7.79 | 8.15 | 23.4 | 180.7 |
| | δ₁ | 7.25 | 7.27 | 7.276 | 7.8 | 3.3 |
| | ε₁ | 10.08 | 10.10 | 10.13 | 9.4 | 14.6 |
| | ε₃ | 7.43 | 7.45 | 7.43 | 10.1 | −14.2 |
| | ζ₂ | 7.69 | 7.71 | 7.68 | 12.9 | −17.7 |
| | ζ₃ | 7.22 | 7.22 | 7.23 | 1.1 | 5.5 |
| | η₂ | 7.17 | 7.18 | 7.16 | 4.8 | −6.5 |

Interestingly, although the Δδₐ values of the δ₂ for both histidines are similar, the Δδₐ of Hε₁ and of H_{ß} for the first histidine (His2) are 3 times higher than the values for the last histidine (His3) (Table 1). This difference may indicate that the complex involves two different tautomeric forms of histidine in the peptide's coordination to the Zn²⁺ ion. The deshielding effect for His2 may arise from the lower electron density around these protons because of their proximity to the metal. This suggests that the zinc coordination is carried out through Nδ₁ for His2 and through N_{ε2} for His3.

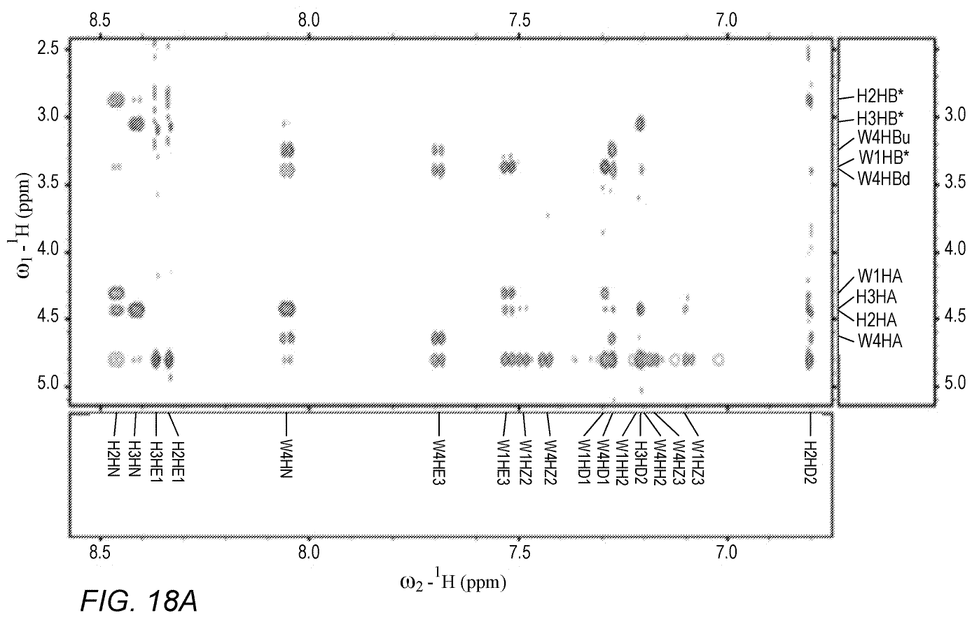

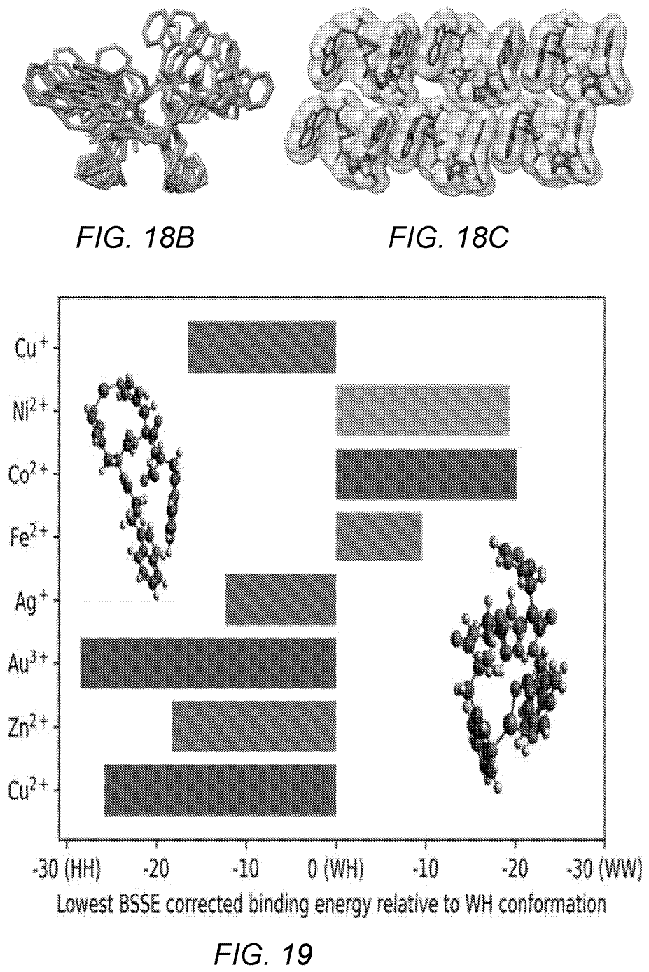

2D ¹H-NMR correlated spectroscopy (COSY), total correlation spectroscopy (TOCSY), and rotating frame Overhauser enhancement spectroscopy (ROESY) measurements were performed to determine the structure of the peptide and of the complex under aqueous and emulsion conditions. The spectra were assigned according to Wüthrich and used to create ensembles of low energy conformations of the peptide (FIGS. 10B-D). In the case of the free peptide, the histidines pointed to opposite directions (FIG. 10B, RMSD values: Backbone 0.86 Å, heavy atoms 2.36 Å, heavy atoms of His residues 1.39 Å). After adding 1 molar equivalent of Zn²⁺ ions, the histidines moved closer in order to coordinate the metal (FIGS. 10C and 17B). A similar observation was derived from all-atom simulations of the peptide with and without Zn²⁺ ions, where the average dihedral angle was reduced from 50-60° to 0-10° upon addition of the metal. The structure of the ensemble was calculated based on the binding information derived from deviations in chemical shift upon binding (FIG. 10C, RMSD values: Backbone 0.78 Å, heavy atoms 2.13 Å, heavy atoms of His residues 0.77 Å). The structure displayed freedom of motion in the tryptophan side chains. Contrarily, the ensemble determined for the Zn²⁺-bound peptide in the emulsion displayed a significantly more confined structure where the tryptophan side chains also showed limited motion (FIG. 10D, RMSD values: Backbone 0.32 Å, heavy atoms 0.71 Å, heavy atoms of His residues 0.04 Å). This constraint could originate from aromatic and hydrophobic intermolecular interactions between neighboring peptides at the CDCl₃-water interface. As hypothesized in FIG. 10E, the peptides may organize at the interface with the C-terminus of one peptide facing the N-terminus of the other peptide, forming salt bridge interactions in addition to the aromatic and hydrophobic interactions of the indoles (FIG. 18).

### Delivery of Anti-Cancer Drugs

The ability to use these emulsions for drug delivery purposes was assessed using the 3-(4,5-dimethylthiazol-2-yl)-2,5-diphenyltetrazolium bromide (MTT) assay. The assay was conducted on cervical adenocarcinoma HeLa cells with the widely used drug paclitaxel (PTX). For clinical use, PTX is usually dissolved in Kolliphor® EL (polyethoxylated castor oil, formerly Cremophor® EL) and ethanol, due to its low solubility. This poses significant disadvantages due to stability issues of the drug that can precipitate when diluted, causing therapeutic failures and possibly harming the patients. Life-threatening hypersensitivity reactions to the vehicle are also possible, ranging from mild effects to systemic anaphylaxis, which can prove fatal. One way to overcome these complications is to administer the drug in an emulsion. An additional advantage of the system proposed herein is that the disassembly of the emulsion droplets will release the metal ions of the complex in addition to the PTX. Since the efficacy of PTX is increased in the presence of Zn²⁺ ions, their release in proximity to the drug could enhance its therapeutic potential without introducing stability complications for the drug or the ions.

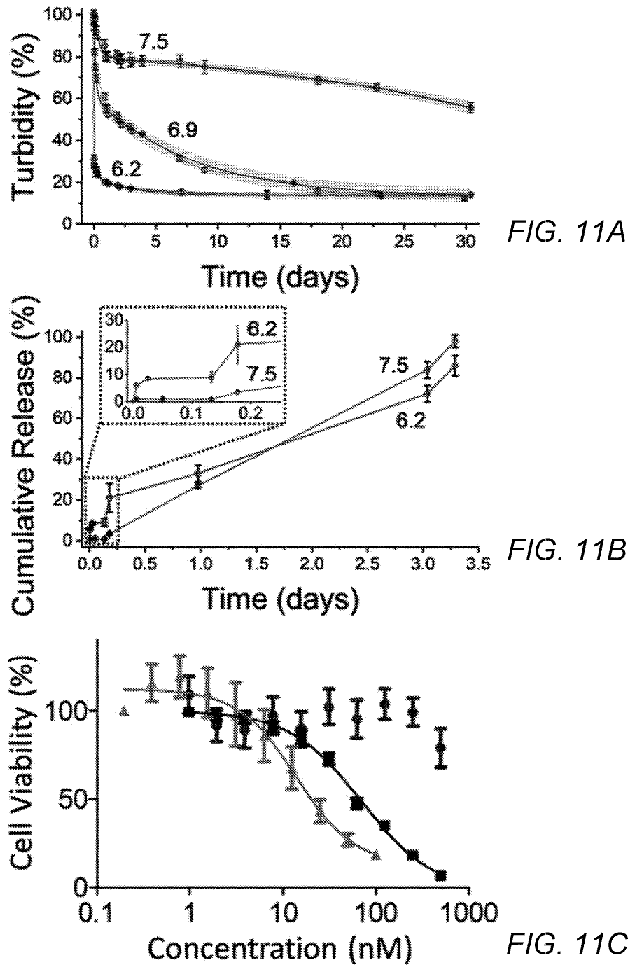

A crucial part of anti-cancer drug delivery through emulsions is their selectivity to cancer cells. The extracellular pH of tumors is found to be 6.2-6.9, which is lower than that of normal tissues (7.2-7.5). The use of the histidine residues as the trigger for disassembling the emulsions and releasing the drugs is therefore beneficial due to the pKₐ of the imidazole's amine (~6). When the emulsion droplets approach the tumor, their stability will decrease, and they will release the drugs in a more selective fashion at the vicinity of the tumor. This behavior can be observed in FIG. 11A by turbidity measurements that can indicate the stability of the emulsion at different pH values. At pH 7.5, the emulsion was stable for several days, dropping below 60% turbidity after 1 month. At pH 6.9 the turbidity dropped below 50% after 2 days, and at pH 6.2 the emulsion completely disassembled after several minutes. The release pattern at pH 7.5 and 6.9, which can be inferred from these measurements appears to be characterized by an immediate burst release followed by a slow-release period. However, at pH 6.2 the burst appears to be absolute, releasing the emulsion contents at once. This was further demonstrated in microscopy images as the emulsion remained stable for more than 1 h at pH values 6.9-7.5, whereas at pH 6.5 the emulsion completely disassembled after 10 min, and the emulsion droplets burst immediately in contact with the buffer at pH 6.2 (FIG. 9).

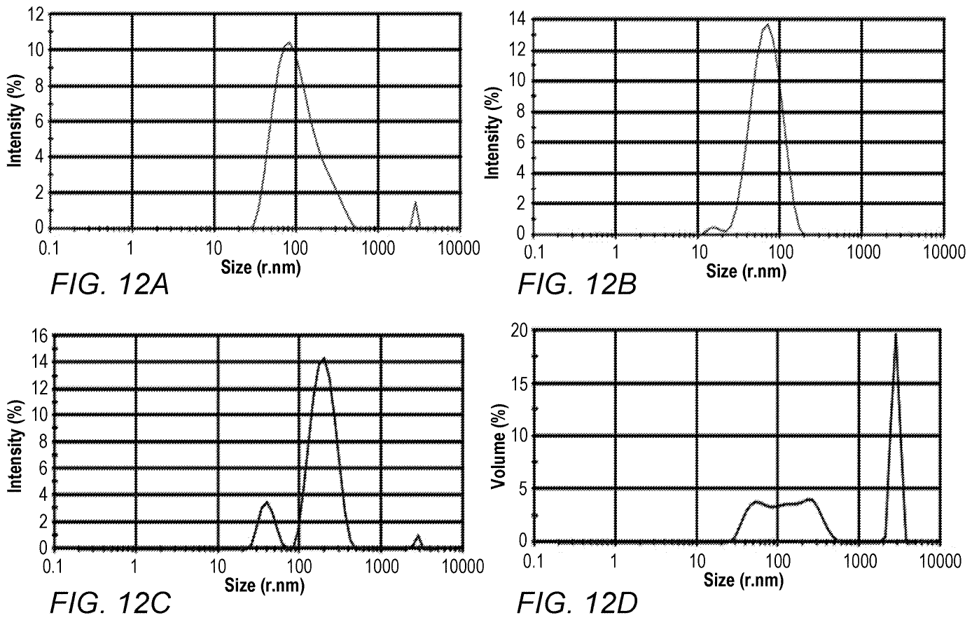

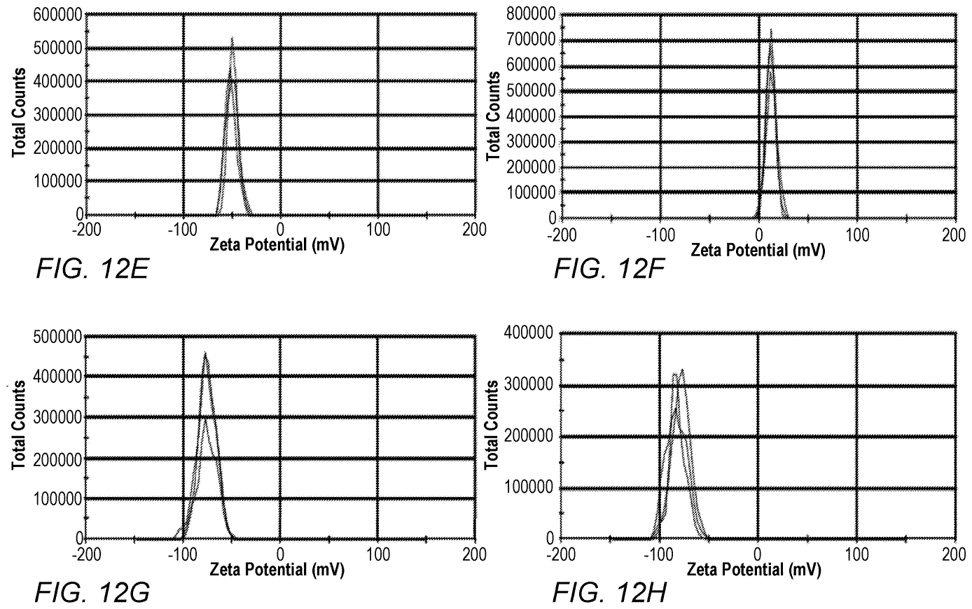

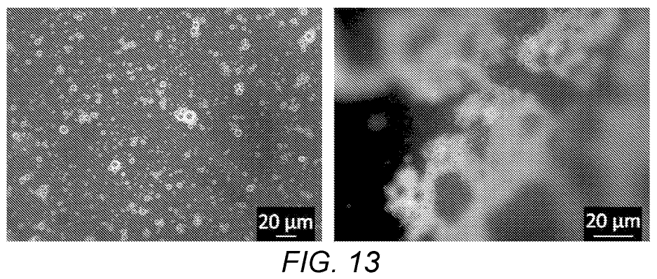

For the drug delivery treatment of cancer cells, the size of the emulsion droplets was reduced by sonication. The size distribution of the emulsion as derived from dynamic light scattering (DLS) was centered at 120 nm, with a small fraction (1.6%) of droplets at the size of 2750 nm (FIG. 12A). While it is possible that the smaller droplets may enter the cells, the release of the drugs outside of the cells is advantageous as it increases the selectivity of the emulsion to cancer cells by bursting specifically at the lower extracellular pH of the tumor cells. Nonetheless, it is possible to filter out the larger droplets and further steps may be taken to affect the size of the droplets (FIG. 12B).

The emulsion itself did not display any cytotoxicity (FIG. 11B). When the drug was tested on its own, it showed strong cytotoxic activity with a half maximal inhibitory concentration (IC50) of 14±5 nM. This is in accordance with the known values for this drug. The results for PTX encapsulated in the emulsion were slightly higher at 70±10 nM. The decrease in the activity is expected, as it is possible that not all of the drug was encapsulated inside the droplets. Additionally, the drug may remain in the less dense castor oil phase after the emulsion disassembly, decreasing its contact with the cells attached to the bottom of the plate.

To assess the capability of the peptide-stabilized emulsion to deliver other drugs we measured the encapsulation efficiency (% EE) of seven additional compounds: ampicillin, disulfiram, piperine, riboflavin, FITC, nile red, and oil red O. The first four of these compounds were reported to exhibit anti-tumor properties among additional medicinal uses. While the size of the compound did not appear to affect the encapsulation efficiency, the compounds' solubility in the oil phase appears to be the determining factor, as can be inferred from the log P values (Table 2). The lower % EE values for FITC and oil red O occurred due to their low solubility in castor oil. The % EE values may be altered to fit different drugs by changing the oil phase or by changing the emulsion preparation parameters such as the oil-water ratio.

TABLE 2
Encapsulation efficiency of various compounds

| | Molecular | Solubility in | | % |
|---|---|---|---|---|
| | weight (g/mol) | water (mg/L) | logP | EE |
| Ampicillin | 349.406 | 10,100 (21° C.)^{a} | 1.35ª | 40 ± 3 |
| Oil red O | 408.51 | Insoluble | 7.58d | 43 ± 5 |
| Riboflavin | 376.36 | 84.7 (25° C.)^{a} | −1.46ª | 51 ± 3 |
| FITC | 389.382 | Insoluble | 4.8^{b} | 62 ± 9 |
| Nile red | 318.369 | Insoluble | 3.8^{b} | 95.0 ± 0.9 |
| Disulfiram | 296.539 | 4.09 (25° C.)^{a} | 3.88ª | 96.2 ± 0.8 |
| Paclitaxel | 853.906 | Insoluble | 3ª | 97.8 ± 0.3 |
| Piperine | 285.35 | 40 (18° C.)^{c} | 3.5^{b} | 98.3 ± 0.4 |

Values for the encapsulation efficiency (% EE) of various compounds inside emulsions using castor oil as the non-aqueous phase.
ªValues taken from DrugBank. Predicted values (XlogP3-AA).
^{b}Values taken from DrugBank.
^{c}Predicted values (ACDLabs).

Overall, these results demonstrate that this peptide-stabilized emulsion can be used for the delivery of drugs such as the delivery of the water-insoluble PTX alongside Zn²⁺ ions, with higher selectivity towards the tumor environment.

### A Multifunctional Drug Delivery System

As mentioned above, delivery via emulsions is mostly limited to the encapsulated material. The ability to easily coat emulsions droplets with different compounds by demand can greatly expand the potential of this drug delivery system. This aim was investigated with the use of the simple and common metal-binding moiety hexahistidine. This sequence is frequently used in protein purification as it can coordinate to different metal ions including Cu²⁺ and Zn²⁺. Many different compounds are isolated with a hexahistidine tag, and more compounds are commercially available. Aside from protein purification, hexahistidine tags can be utilized for various applications. By simply mixing the emulsion with hexahistidine-tagged compounds, the emulsion droplets can be functionalized for various purposes.

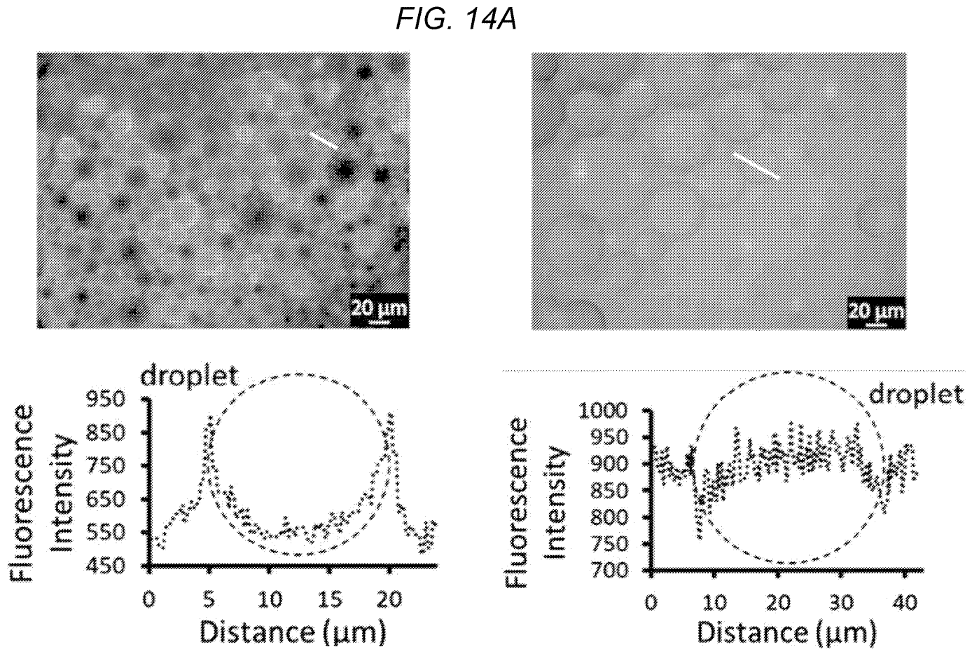

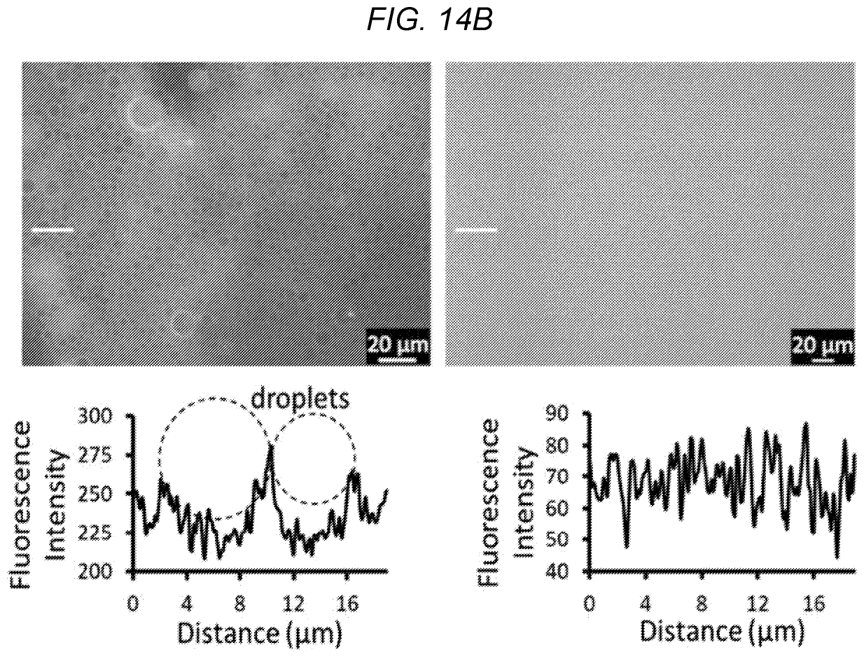

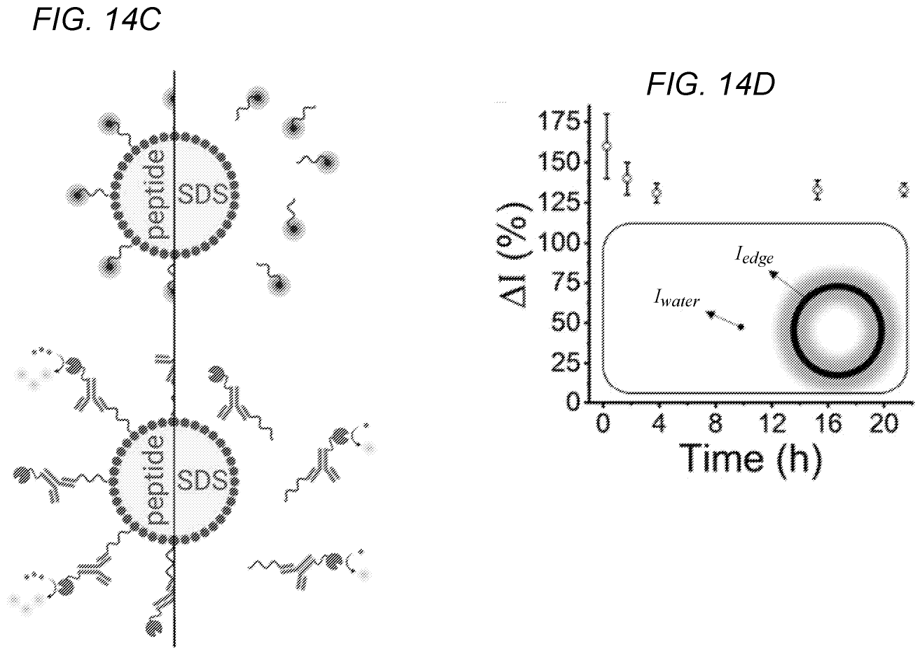

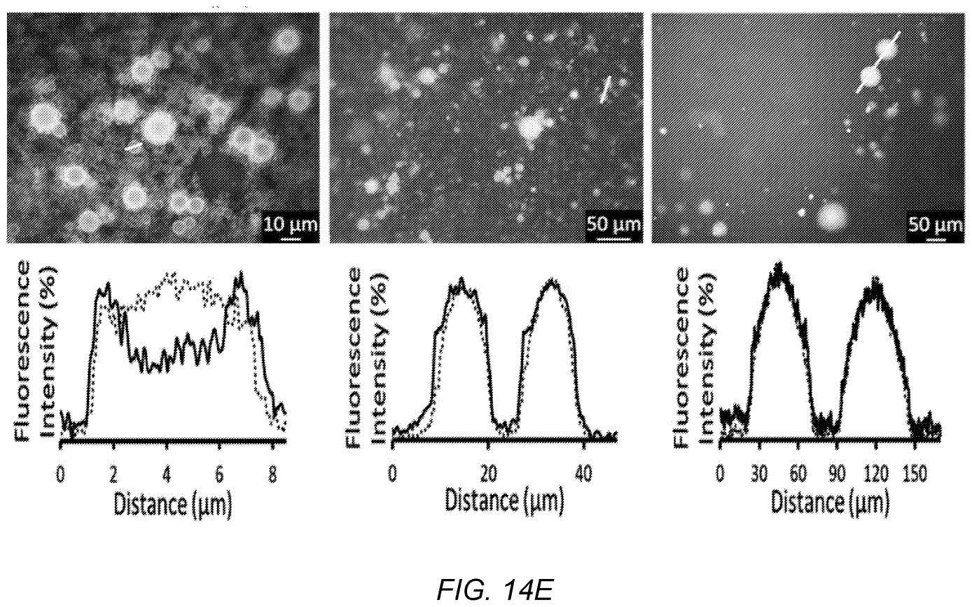

To prove this concept, we synthesized a hexahistidine peptide labeled with Rhodamine B (H₆—Rh). The coating of the emulsion droplets with this peptide was demonstrated by adding it to emulsions formed with the complex and with SDS at the same concentration as a control (FIG. 14A). The stronger fluorescence at the circumference of the droplets indicates the binding of the hexahistidine groups to the free coordination sites of the metal ions in the complex. In contrast, the SDS-stabilized emulsions did not display fluorescence surrounding the droplets, indicating the absence of the fluorescent peptide on the surface of the droplets.

To further widen the proof of concept, we utilized the horseradish peroxidase (HRP)-luminol system. In this system, a hexahistidine-tagged antibody conjugated to the enzyme HRP catalyzes the oxidation of luminol in presence of H₂O₂, bringing it to an excited state that produces fluorescence. In a similar manner, the hexahistidine-tagged antibody was mixed with the peptide-stabilized emulsion and with an SDS-stabilized emulsion at the same concentration of the surfactant. When the reagents which include the H₂O₂ and luminol were added, the fluorescence was visible at the vicinity of the peptide-stabilized emulsion droplets, indicating the binding of the hexahistidine groups to the metal ions of the complex (FIG. 14B). Contrarily, with the SDS-stabilized emulsion, the fluorescence was weak and not localized at the droplets. This indicates that the enzyme did not bind the droplets and remained free in the solution, forming excited luminol that was dispersed in a much lower concentration at the aqueous solution.

These results were confirmed using ζ-potential measurements. The ζ-potential values for the emulsions with Zn²⁺ or with Cu²⁺ ions were −51±1 mV and −75.3±0.8 mV, respectively (FIG. 12). After addition of ~0.2 eq. of the H₆—Rh peptide to the emulsion with Zn²⁺ ions, the charge changed to −11.9±0.3 mV, in accordance with the positive charge of the rhodamine. After addition of 0.2 μg/mL of the HRP probe antibody to the emulsion with Cu²⁺ ions, the charge changed to −81±2 mV. This is also expected due to the overall negative charge of HRP at pH≥5.54. The coordinate bond between the hexahistidine-tagged compounds and the metal on the emulsion's surface appears to be stable for up to 24 h (FIG. 14D). However, it is important to mention that the compound chosen to decorate the emulsion droplets may induce agglomeration due to the change in the ζ-potential of the droplets (FIG. 13).

The ability of the emulsion to simultaneously release hydrophobic and hydrophilic compounds was demonstrated by encapsulating nile red inside the oil phase and decorating the droplets with a hexahistidine peptide labeled with FITC (H₆-FITC). At pH 7.5, the red fluorescence of nile red can be observed inside the oil droplets and the green fluorescence of the FITC-labeled peptide can be seen encircling the droplets (FIG. 14E). When the pH is lowered, the emulsion disassembled, the oil droplets began to coalesce, and the H₆-FITC peptide formed aggregates. At acidic pH, large oil droplets can be observed, with no localization of the green fluorescence derived from the H6-FITC peptide.

Overall, the ability to simply decorate the emulsion droplets could provide them with numerous unique functions: attaching peptides or proteins may be used for the delivery of hydrophilic peptide or protein therapeutics; labeling the emulsion droplets with fluorescent dyes may be used for tumor imaging; emulsions coated with antibodies may mediate their targeting to specific locations.

### CONCLUSIONS

We described herein a peptide that can be used to stabilize emulsions. These emulsions can be used as an intricate drug delivery system with the ability to deliver lipophilic compounds encapsulated inside the droplets, metal ions bound to the peptide, and hydrophilic compounds attached to the metal ions on the surface of the droplets. The unique complexation-triggered amphiphilicity of the peptide and the emulsions it forms could be applied for a multitude of drug delivery purposes, which were previously difficult to achieve.

### EXPERIMENTAL PROCEDURES

### Methods

### Materials

2-Chlorotrityl chloride resin (1.0-1.6 mmol g⁻¹, 100-200 mesh) and N,N′-Diisopropylcarbodiimide (DIC) were purchased from Chem-Impex International, Inc. (Wood Dale, IL, United States). 1-[Bis(dimethylamino)methylene]-1H-1,2,3-triazolo[4,5-b]pyridinium 3-oxide hexafluorophosphate (HATU) was purchased from Matrix Innovation, Inc. (Saint-Hubert, QC, Canada). Triisopropylsilane (TIPS) and nile red were purchased from Tokyo Chemical Industry (Tokyo, Japan). Dimethylformamide (DMF), dichloromethane (DCM), piperidine, methanol, diethyl ether, N,N-Diisopropylethylamine (DIEA), acetonitrile, chloroform, and trifluoroacetic acid (TFA) were purchased from Bio-Lab (Jerusalem, Israel). Fmoc-Trp (Boc)-OH and the peptides Phe-His-His-Phe, Tyr-His-His-Tyr, Ala-His-His-Ala, Trp-Ala-Ala-Trp, and Trp-His were purchased from GL Biochem (Shanghai, China). EZ-ECL Enhanced Chemiluminescence Detection Kit for HRP was purchased from Biological Industries (Beit Haemek, Israel). His-probe (H-3) HRP sc-8036 was purchased from Santa Cruz Biotechnology (Dallas, TX, United States). Hydrogen tetracholoroaurate (III) hydrate, copper (I) chloride, and copper (II) chloride were purchased from Acros Organics (Fair Lawn, NJ, United States). Silver nitrate 99+%, castor oil, fluorescein isothiocyanate (FITC), and Fmoc-His(Trt)-OH were purchased from Alfa Aesar (Lancashire, United Kingdom). Zinc (II) chloride, histidine, tryptophan, oil red O, ampicillin, and paraffin oil were purchased from Merck (Rahway, NJ, United States). Deuterated chloroform was purchased from Cambridge Isotope Laboratories (Tewksbury, MA, United States). Rink amide resin (0.48 mmol g⁻¹) was purchased from Matrix Innovations (Hoddesdon, United Kingdom). Ethyl cyanohydroxyiminoacetate (Oxyma) and hydroxybenzotriazole (HOBt) were purchased from Luxembourg Biotechnologies Ltd. (Rehovot, Israel). Rhodamine B, FITC, riboflavin, disulfiram, and piperine were purchased from Thermo Fisher Scientific (Waltham, MA, United States).

### Peptide Synthesis

NH2-Trp-His-His-Trp-OH (SEQ ID NO 11) was manually synthesized by Fmoc solid phase peptide synthesis on a 2-Chlorotrityl chloride resin (0.25 mmol). The amino acids at 5 eq. were activated by mixing with a DIEA/HATU mixture (4 eq. and 3.9 eq., respectively) for 4 min. The coupling of the amino acids was carried out for 1 h and confirmed using a Kaiser test. The Fmoc protecting groups were removed by mixing with 20% piperidine in DMF for 20 min. The resin was washed between steps twice with DMF, methanol, DCM, and DMF again. The cleavage reaction was performed by mixing with a TFA/TIPS/water mixture (38:1:1) for 3 h. The cleaved solution was evaporated, precipitated with diethyl ether, and centrifuged. The peptide product was dissolved in water and lyophilized.

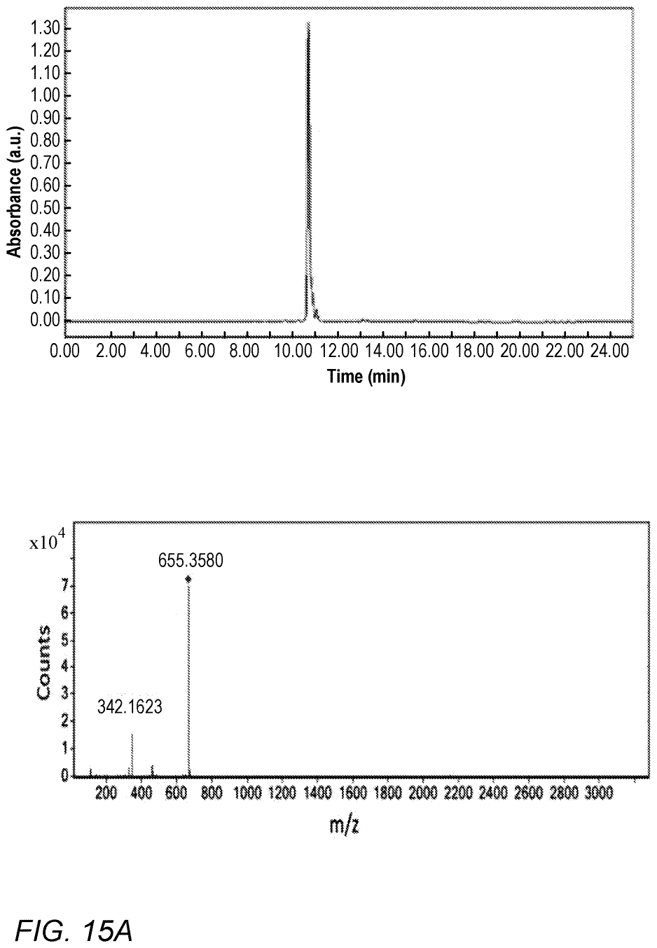

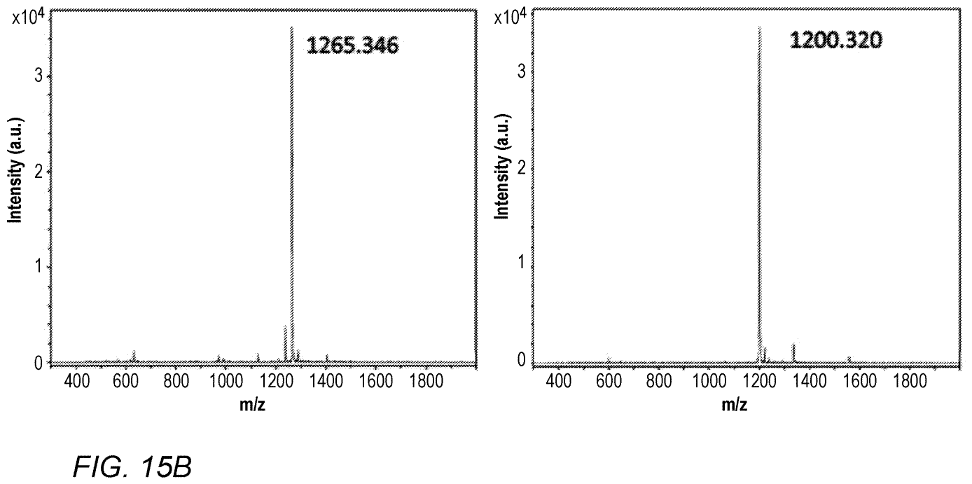

The purity of the peptide was assessed using analytical reverse-phase high-performance liquid chromatography (HPLC) analysis (Waters Alliance) with UV detection (220 nm and 280 nm) and an XSelect C18 column (3.5 μm, 130 Å, 4.6 mm×150 mm) (FIG. 15). The peptide was eluted using a linear gradient of ACN in TDW (0.1% TFA, 1 mL min⁻¹, 30° C.). The mass of the peptide was determined using liquid chromatography mass spectrometry (LC/MS) with Agilent 6520 Q-TOF analyzer (Agilent Technologies, Santa Clara, CA, United States).

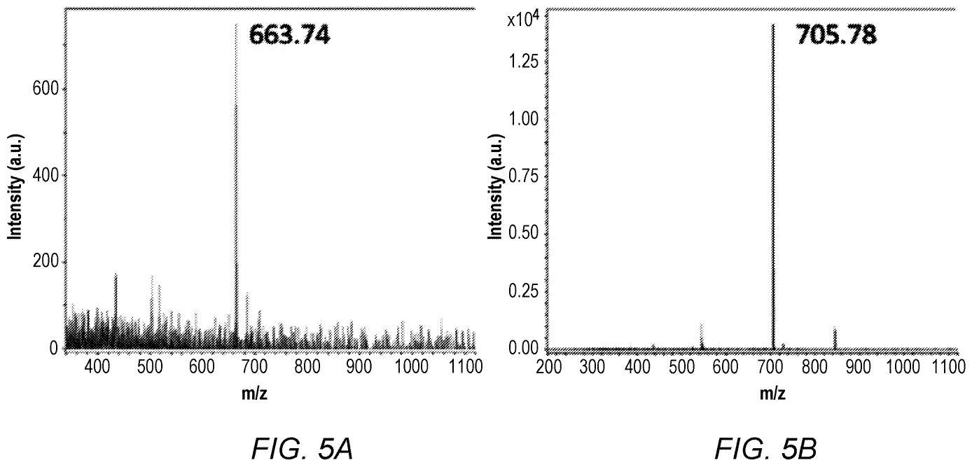

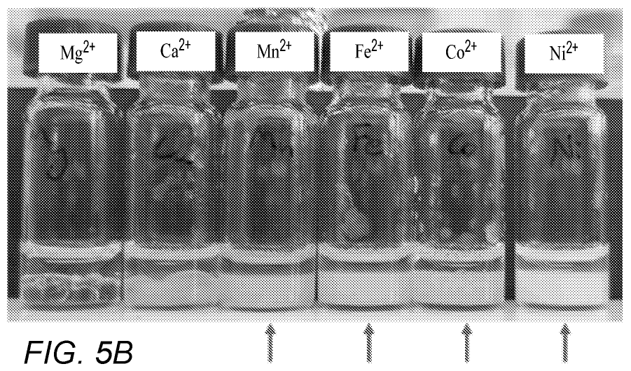

NH₂-(His)₆-Rhodamine B and NH₂-(His)₆-FITC were synthesized on a Liberty Blue Microwave-Assisted Peptide Synthesizer using Fmoc amino acids and Oxyma/DIC/DIEA coupling reagents. The peptides were labeled prior to the cleavage by mixing it with rhodamine B or FITC (100 mg), HOBT (36 mg), and a mixture of DMF/DCM/DIEA/DIC (40.5:4.8:1.1:1) for 3 h in the dark. The peptides' identity and purity were confirmed using MALDI TOF/TOF AutoFlex Speed (Bruker Daltonics Inc., Bremen, Germany) (FIG. 5).

### Emulsion Preparation

The emulsions were prepared by mixing a peptide solution with the metal ions to a final concentration of 1 mm each and adding NaOH to obtain pH=10. The desired non-aqueous phase was added to this solution at 50% (v/v) and the two phases were manually agitated to form the emulsions. For the emulsions with chloroform, it was found that the best procedure was preparing a 50% (v/v) chloroform emulsion and then adding chloroform to 67% (v/v) and manually agitating again. To achieve smaller-sized emulsion droplets with castor oil, the same procedure was performed, following sonication for 1 h in an ultrasonic bath.

### Emulsion Characterization

For fluorescence microscopy, the castor oil was labeled with nile red prior to the emulsification. For labeling the peptide on the emulsion droplets, the prepared emulsion was mixed with FITC (4 μg mL-1) for 1 h. The images were taken using an Axio Scope A1 fluorescence microscope (Zeiss, Oberkochen, Germany).

For the UV-vis titration, a peptide solution (1.5 mm) was titrated with a CuCl₂ solution (15 mM). The solutions were left for 5 min and the absorbance was measured using a UV-vis spectrophotometer (Shimadzu, UV-1650PC, Kyoto, Japan). For the Job's plot assay, peptide solutions with different mole fractions of Cu²⁺ ions (up to 1 mm) were prepared at pH=9 and their absorbance values were measured.

The far-UV CD spectra were recorded with a J-810 spectropolarimeter (JASCO Inc., Easton, MD, United States), using a 0.1 cm pathlength far-UV CD spectroscopy quartz cuvette (between 190-260 nm, 0.1 nm intervals).

The size distribution of the emulsion stabilized with peptide-Zn²⁺ was measured using a Zetasizer ZEN3600 (Malvern instruments, Malvern, UK).

### NMR Studies

The samples were prepared in 20 mm phosphate buffer (pH=6.5, 10% D₂O) with the peptide (3 mM) and Zn²⁺ ions (3 mm) when mentioned. The measurements were taken at 298.1 K. For the emulsion spectra, a solution of zinc chloride (3 mm) was added to the peptide (3 mM). The solution was in phosphate buffer 20 mm, pH 6.5 with 10% D₂O. 100 μL of CDCl₃ was added to form an emulsion, where the excess chloroform settled to the bottom of the test tube below the line of the coils. The titration study was performed by addition of Zn²⁺ ions from 0 to 6 mM (2 equiv) and an addition of 15% (v/v) of CDCl₃.

The NMR experiments were performed on a Bruker AVII 500 MHz spectrometer operating at the proton frequency of 500.13 MHz, using a 5-mm selective probe equipped with a self-shielded xyz-gradient coil at 25° C. The transmitter frequency was set on the water signal. COSY, TOCSY using the MLEV-17 pulse scheme for the spin lock (150 ms), and ROESY experiments (using an optimized mixing time of 400 ms), were acquired using gradients for water saturation under identical conditions.

Spectra were processed and analyzed with TopSpin (Bruker Analytische Messtechnik GmbH) and NMRFAM SPARKY software. Resonance assignment followed the sequential assignment methodology developed by Wüthrich.

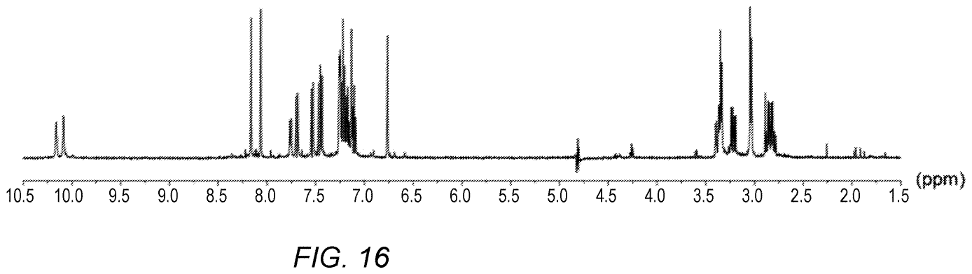

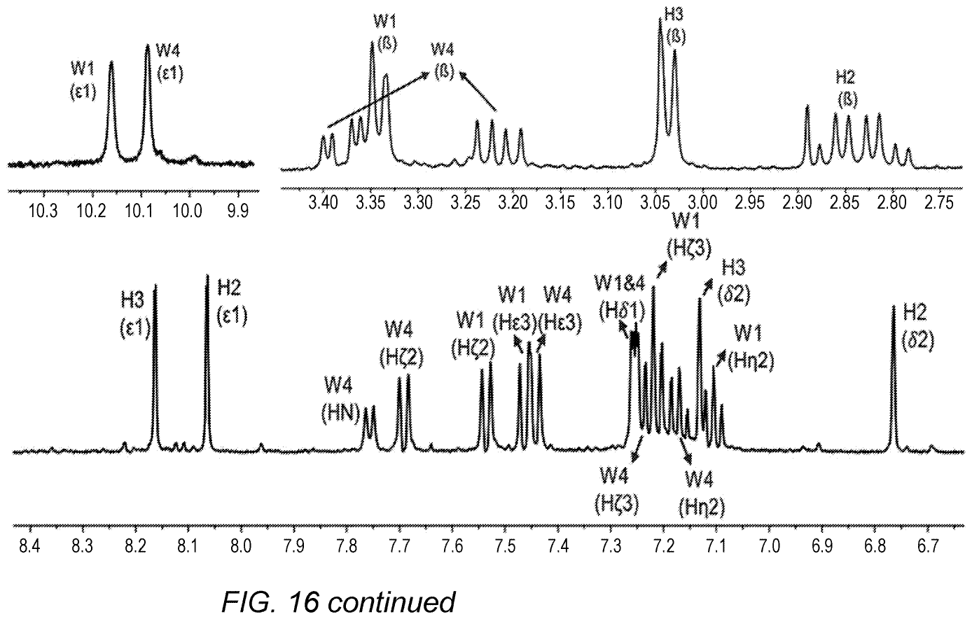

The three-dimensional structures of the peptides were calculated using XPLOR-NIH (version 3.2) by hybrid distance geometry-dynamical simulated annealing. Peak intensities were manually assigned as strong (2.5 Å), medium (3.5 Å), weak (4.5 Å) and very weak (5.5 Å) with a ±0.5 Å error. Parameters were introduced using zinc geometry and charges were derived from the Amber forcefields. Fifty initial structures were generated. The nuclear Overhauser effect (NOE) restraint energy was introduced as a square-well potential. Molmol was used to create the final ensemble of structures. Low energy structures chosen for further analysis had no NOE violations, deviations from ideal bond lengths of less than 0.05 Å, and bond angle deviations from ideality of less than 5°. Figures were produced, using ChimeraX from the University of California, San Francisco (supported by NIH P41 RR-01081) (FIG. 16).

### Molecular Dynamic Simulations

Coarse-grained molecular dynamics (CGMD) was performed using the GROMACS software package using the MARTINI 3 forcefield and a timestep of 20 fs. Velocity rescaling was used to hold the temperature around 310 K and a Berendsen barostat was used to control the isotropic pressure at 1 bar. Bond lengths within aromatic side chains were constrained using the LINCS algorithm. Short range intermolecular interactions were evaluated using a Lennard-Jones algorithm with a shifted cut off at 1.1 nm and electrostatic interactions evaluated using the reaction-field algorithm with a cut off also at 1.1 nm. All the simulations were performed in aqueous conditions using the MARTINI small water (two water molecules to one bead). All systems were minimized using the steepest descent integrator prior to equilibration.

To compare the magnitude of metal ion binding in the metal ion complexes we used DFT to optimize each structure using the ωB97X functional and def2-TZVP basis set with the auxiliary RIJCOSX basis and the CPCM water model. All calculations were performed using the ORCA 5 program. We used the built-in D4 algorithm to evaluate dispersion interactions. The electrostatic interaction energy was calculated according to Equation 1. We minimized the BSSE by calculating a counterpoise-corrected interaction energy.

Equation 1. Formula for calculating electronic interaction energy between two species that form a complex and performing a counterpoise correction.

$${E}_{interaction} = {E}_{complex} - ( {E}_{ligand} + {E}_{metal} )$$

$$BSSE = {E}_{ligand optimitzed} + {E}_{ligand and ghost atoms} + {E}_{metal ghost atoms} - 2 {E}_{ligand} - {E}_{metal}$$

$${E}_{counterpoise - corrected interaction energy} = {E}_{interaction} - BSSE$$

### Cancer Cell Studies

The behavior of the emulsion at different pH values was performed by adding 1 μL of an emulsion stabilized with a peptide-Zn²⁺ complex to a glass slide with 10 μL of the phosphate buffers (10 mM) at different pH values (FIG. 9).

The turbidity measurements were performed in triplicates after dilution of 10 μL of the emulsion with 690 μL of the phosphate buffers and measuring the turbidity at 350 nm using a Biotek Synergy H1 plate reader (Lumitron), during several days. The emulsions were kept at 37° C. between the measurements. The data were fitted to an exponential decay equation and the 95% prediction band is depicted.

The complete cell-culture medium was prepared by the addition of 10% fetal bovine serum (FBS), 1% L-glutamine, and 1% Penicillin-Streptomycin (all purchased from Sartorius) to Dulbecco's Modified Eagle's Medium (DMEM, Sigma Aldrich). Cervical adenocarcinoma HeLa (American Type Culture Collection) cancer cell lines were cultured as monolayers in complete cell culture medium at 37° C. in a 5% CO₂ atmosphere. The cells were seeded in 96-well plates at a density of ca. 10,000 cells per well and allowed to attach overnight. On the following day, the tested emulsions were prepared at a 10 μM concentration in water, serially diluted to obtain a 10-concentration gradient, and added to the cells for a 72-h incubation. PTX was dissolved in DMSO to a 20 μM concentration, serially diluted in DMSO to obtain a 10-concentration gradient, consequently diluted 10-fold in medium to ensure a final concentration of 0.5% DMSO, and then added to the cells for 72 h. Afterwards, cytotoxicity was measured by the previously reported MTT method. In short, MTT was added to the wells (0.1 mg in 20 μL) for an additional 3 h incubation period. The medium was removed and replaced with isopropanol (200 μL), and upon complete dissolution of the formazan, the absorbance was measured at 550 nm on a Spark 10 M multimode microplate reader spectrophotometer (Tecan Group Ltd., Mannedorf, Switzerland). Each measurement was repeated at least 3×3 times: three repeats per plate, all repeated on at least three different days, giving at least nine repetitions for each experiment. The results for the highest diluted samples of PTX dissolved in DMSO were normalized to 100% cell viability. Relative IC₅₀ values and the standard error of means were determined by a nonlinear regression of a variable slope (four parameters) model using the GraphPad Prism 5.0 software.

The encapsulation efficiency (% EE) values were determined by comparing the amount of the free compound in the aqueous phase with the initial concentration. The compounds were dissolved in castor oil with 10% (v/v) ethanol. The emulsions containing the compounds were centrifuged at 12000 rpm for 10 min and the amount of the compound in the aqueous phase was determined by HPLC (for PTX, disulfiram, ampicillin, and piperine) or by fluorescence measurements (for FITC, nile red, riboflavin, and oil red O). The % EE values were calculated using the formula: % EE=(Cᵢₙᵢₜᵢₐₗ−C_{(aq.)})/Cᵢₙᵢₜᵢₐₗ×100%, where Cᵢₙᵢₜᵢₐₗ is the concentration of the compound in the emulsion and C_{aq.} is the concentration of the compound in the aqueous phase after the centrifugation. The measurements were performed in triplicates.

### Emulsion Functionalization

Coating the emulsion droplets with the fluorescent H₆—Rh peptide was performed by diluting 1 μL of an emulsion stabilized with the peptide-Zn²⁺ complex with 9 μL water and adding the H₆—Rh at a concentration of 0.2 mg mL⁻¹. After 1 h, a drop from the solution was placed on a glass slide and the images were taken using the fluorescent microscope. The same procedure was followed for an SDS-stabilized emulsion and for the H6-FITC-stabilized emulsion.

For the coating with the HRP probe, 1 μL of the HRP probe (20 μg mL⁻¹) was added to 9 μL of a peptide-Cu²⁺-stabilized emulsion with castor oil. The solution was left at room temperature for 1 h and then a drop of 2 μL was placed on a glass slide. To this drop, 5 μL of a 1:1 mixture of both reagents from the HRP probe kit (which include the luminol and the H₂O₂) were added and the images were taken using the fluorescent microscope. The same procedure was followed for an SDS-stabilized emulsion.

The ζ-potential values of the emulsion samples were measured using a Nano-Zetasizer ZEN3600 (Malvern instruments, Malvern, UK) and folded capillary cells (DTS1070).

The stability of the H6-FITC functionalization was determined by fluorescence measurements of the decorated emulsion at different time intervals. The difference in the fluorescence was calculated according to the equation: ΔI=I_{edge}/I_{(aq.)}, where I_{edge} is the fluorescence intensity at the perimeter of the droplets and I_{aq.} Is the fluorescence intensity of the aqueous phase surrounding the droplets. The measurements were performed in triplicates.

1.-93. (canceled)

94. A water-oil mixture comprising a lipophilic agent, a metal-based hydrophilic agent and a reversible surfactant comprising at least one metal-binding moiety and at least one hydrophobic functionality,
the reversible surfactant being switchable between an amphiphilic conformation and a non-amphiphilic conformation, whereby association between the at least one metal-binding moiety of the reversible surfactant and a metal ion of the metal-based hydrophilic agent induces emulsification of both the lipophilic agent and the metal-based hydrophilic agent,
wherein dissociation of the metal-based hydrophilic agent from the reversible surfactant triggers loss of amphiphilicity and release of the hydrophilic and lipophilic agents.

95. The mixture according to claim 94, being an emulsion wherein the at least one metal-binding moiety of the surfactant associates said metal-based hydrophilic agent at an interface between an aqueous phase comprising the metal-based hydrophilic agent and an oil phase comprising or being the lipophilic agent.

96. An emulsion comprising a lipophilic agent, a metal-based hydrophilic agent and an amphiphilic surfactant comprising at least one metal-binding moiety and at least one hydrophobic functionality,
the at least one metal-binding moiety of the amphiphilic surfactant associating said metal-based hydrophilic agent at an interface between an aqueous phase comprising the metal-based hydrophilic agent and an oil phase comprising or being the lipophilic agent, wherein dissociation of the metal-based hydrophilic agent from the amphiphilic surfactant triggers loss of amphiphilicity and release of the hydrophilic and lipophilic agents.

97. The emulsion according to claim 96, wherein the dissociation is triggered by a change in pH, a change in ionic strength, or a change in temperature.

98. The emulsion according to claim 94, wherein the surfactant is a peptide.

99. The emulsion according to claim 98, wherein the peptide comprises one or more metal-binding moieties capable of associating to a metal ion of monovalent, bivalent, trivalent and higher oxidation metals.

100. The emulsion according to claim 95, wherein the metal-binding moiety is capable of reversible associate to a metal ion.

101. The emulsion according to claim 100, wherein the surfactant is a peptide and wherein the metal-binding moiety is a nitrogen group of an amino acid.

102. The emulsion according to claim 94, wherein the surfactant is a peptide comprising two or more histidine amino acids and one or more hydrophobic amino acid.

103. The emulsion according to claim 94, wherein the one or more hydrophobic functionality is a hydrocarbon having at least 6 carbon atoms, an aromatic group or a large chemical group having low or no solubility in water.

104. The emulsion according to claim 103, wherein the hydrophobic functionality is a hydrophobic amino acid.

105. The emulsion according to claim 94, wherein the surfactant is a peptide selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4).

106. The emulsion according to claim 95, wherein the surfactant is a peptide, wherein the metal-binding moiety is histidine and the hydrophobic functionality is phenylalanine or naphthylalanine.

107. A surfactant switchable between amphiphilic and non-amphiphilic conformations, the surfactant comprising one or more metal-binding moieties and one or more hydrophobic functionalities, wherein binding of a metal ion to the one or more metal-binding moieties triggers a reversible conformational change from the non-amphiphilic conformation to the amphiphilic conformation.

108. A method for (simultaneous) delivery or for rendering available a combination of a lipophilic agent and a metal-based hydrophilic agent to a medium, the method comprising
contacting the medium or administering to the medium or bringing in contact with the medium an emulsion comprising a surfactant according to claim 107, the surfactant being provided in an interface formed between the lipophilic agent and an aqueous phase, wherein the surfactant is chemically associated with a metal of the metal-based hydrophilic agent; and
causing dissociation of the metal of the metal-based hydrophilic agent to thereby deliver or render available to the medium both the lipophilic agent and the metal-based hydrophilic agent.

109. The method according to claim 108, wherein the surfactant is a peptide.

110. A reversible surfactant being a peptide selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4).

111. A water-oil mixture comprising a lipophilic agent, a metal-based hydrophilic agent and a reversible surfactant selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4),
the reversible surfactant being switchable between an amphiphilic conformation and a non-amphiphilic conformation, whereby association with a metal ion of the metal-based hydrophilic agent induces emulsification of both the lipophilic agent and the metal-based hydrophilic agent,
wherein dissociation of the metal-based hydrophilic agent from the reversible surfactant triggers loss of amphiphilicity and release of the hydrophilic and lipophilic agents.

112. An emulsion comprising a lipophilic agent, a metal-based hydrophilic agent and an amphiphilic surfactant selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4),
the at least one metal-binding moiety of the amphiphilic surfactant associating said surfactant at an interface between an aqueous phase comprising the metal-based hydrophilic agent and an oil phase comprising or being the lipophilic agent, wherein dissociation of the metal-based hydrophilic agent from the amphiphilic surfactant triggers loss of amphiphilicity and release of the hydrophilic and lipophilic agents.

113. A non-amphiphilic peptide selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4), said peptide being reversibly convertible into an amphiphilic compound upon exposure to metal ions.

114. A surfactant switchable between amphiphilic and non-amphiphilic conformations, the surfactant being selected from Trp-His-His-Trp (SEQ ID NO: 1), Trp-His-Trp-His (SEQ ID NO: 2), His-Trp-Trp-His (SEQ ID NO: 3), and His-Trp-His-Trp (SEQ ID NO: 4), wherein binding of a metal ion to the surfactant triggers a reversible conformational change from the non-amphiphilic conformation to the amphiphilic conformation.
